SEQUENCE LISTING (6 sequences)

SEQ ID NO: 1 - protein, 1815 aa, Neisseria meningitidis
MKTKRFKINAISLSIFLAYALTPYSEAALVRDDVDYQIFRDFAENKGKFFVGATDLSVKNKQGQNIGNALSNVPMIDFSVADVNRRTLTVIDPQYAVSVKHVKGDEISYYGHHNGHLDVSNDENEYRSVAQNDYEPNKNWHHGNQGRLEDYNMARLNKFVTEVAPIAPTSAGGGVETYKDKNRFSEFVRVGAGTQFEYNSRYNMTELSRAYRYAIAGTPYQDVNVTSNLNQEGLIGFGDNSKHHSPEKLKEVLSQNALTNYAVLGDSGSPLFAYDKQEKRWVFLGAYDYWAGYQKNSWQEWNIYKKEFADEIKQRDNAGTIKGNGEHHWKTTGTNSHIGSTAVRLANNERDANNGQNVTFENNGTLVLDQNINQGAGGLFFKGDYTVKGINNDITWLGAGIDVADGKKVVWQVKNPNGDRLAKIGKGTLEINGTGVNQGQLKVGDGTVILNQQADADKKVQAFSQVGIVSGRGTLVLNSSNQINPDNLYFGFRGGRLDANGNDLTFEHIRNVDEGARIVNHNTGHASTITLTGKSLITDPKTISIHYIQNNDDDDAGYYYYRPRKPIPQGKDLYFKNYRYYALKSGGSVNAPMPENGQTENNDWILMGSTQEEAKKNAMNHKNNQRISGFSGFFGEENGKGHNGALNLNFNGKSAQNRFLLTGGTNLNGKISVTQGNVLLSGRPTPHARDFVNKSSARKDAHFSKNNEVVFEDDWINRTFKATEIAVNQSASFSSGRNVSDITANITATDNAKVNLGYKNGDEVCVRSDYTGYVTCNTGNLSDKALNSFGATQINGNVNLNQNAALVLGKAALWGQIQGQGNSRVSLNQHSKWHLTGDSQVHNLSLADSHIHLNNASDAQSANKYHTLKINHLSGNGHFHYLTHLAKNLGDKVLVKESASGHYQLHVQDKTGEPNQEGLNLFDASSVQDRSRLSVSLANNHVDLGALRYTIKTENGITRLYNPYAENRRRVKPAPSPATNTASQAQKATQTDGAQIAKPQNIVVAPPSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARRHEKEREAAELSAKQRVGEEERRQTAQSQPQRRKRRAAPQDYMAASQDRPKRRGHRSVQQNNVEIAQAQAELARRQQEERKAAELLAKQRAEAEREAQALAARRKAEAEEAKRQAAELAHRQEAERKAAELSANQKAAAEAQALAARQQKALARQQEEARKAAELAVKQKAETERKTAELAKQRAAAEAAKRQQEARQTAELARRQEAERQAAELSAKQKAETDREAAESAKRKAEEEEHRQAAQSQPQRRKRRAAPQDYMAASQNRPKRRGRRSTLPAPPSPSFDSSAYAAPRALHNPDWYENDYEEIPLDALEDENVSESVDTSDKQPQDNTELHEKYENDYEEIPLDALEDEDVSESVDTSDKQPQDNTELHEKVETVSLQPRAAQPRAQAATQLQAQAAAQADAVSTNTNSALSDAMASTQSILLDTGASLTRHIAQKSRADAEKNSVWMSNTGYGRDYASAQYRRFSSKRTQTQIGIDRSLSENMQIGGVLTYSDSQHTFDQASGKNTFVQANLYGKYYLNDAWYVAGDIGAGSLRSRLQTQQKANFNRASIQTGLTLGNTLKINQFEIVPSAGIRYSRLSSADYKLGNDSVKVSSMSVKTLTAGLDFAYRFKVGNLTVKPLLSAAYFANYGKGGVNVGGNSFVYKADNQQQYSAGAALLYRNVTLNVNGSITKGKQLEKQKSGQIKIQIRF

SEQ ID NO: 2 - DNA, 5448 bp, Neisseria meningitidis
  source 1..5448: /mol_type=“unassigned DNA” /organism=“Neisseria meningitidis”
atgaaaaccaaacgttttaaaattaacgccatatccttatccatctttcttgcctatgcccttacgccatactcagaagcggcattggtcagagacgatgtcgattatcaaatattccgtgactttgcagaaaacaaaggcaaattttttgtcggcgcaaccgatttatcagtgaaaaacaaacaaggtcaaaacatcggcaacgcattatccaacgtaccgatgattgattttagcgttgcagatgtcaacagacgtacattaaccgtcatcgatccccagtatgccgtcagcgtcaaacatgtaaaaggagacgaaatctcttattacgggcatcacaatgggcacttagatgttagcaatgatgaaaacgaatatcgttcagttgcacaaaatgactacgaaccaaataaaaattggcatcacggtaatcaaggtcgtcttgaagactacaacatggcacgccttaataaattcgtaacagaagtcgcacctattgcaccaaccagtgcaggcggcggcgttgaaacctataaagataaaaaccgtttttctgagtttgtgcgagttggagcaggtacgcaatttgaatataacagccgctacaatatgacagagctttcacgagcttatcgttatgccattgcaggtacgccttatcaagacgtgaatgtcacatcgaatctaaatcaagaaggcttgattggcttcggtgataattcaaaacatcattccccagaaaaactcaaagaagttttatctcaaaacgcattgactaactatgctgtgttaggcgatagcggttcaccattatttgcttatgataaacaagaaaaacgctgggtctttttaggtgcttacgattattgggcaggctatcaaaaaaactcttggcaagaatggaatatctataaaaaagaatttgcagatgaaatcaaacaacgcgataacgccggcaccatcaaaggtaatggagaacatcattggaaaaccacgggcacaaacagccatatcggttcgacagcggtaaggcttgccaacaatgaaagggatgcaaacaacggacagaatgttacctttgaaaacaacggcactttggtattggatcaaaacatcaaccaaggcgcgggcggtctgtttttcaaaggcgattacacagtcaaaggtataaataatgacatcacttggctaggtgcggggattgatgttgccgacggcaaaaaagtcgtttggcaagtcaaaaatccgaatggcgacagattggcaaaaatcggcaaaggcacattagaaataaacggcacaggcgttaaccaagggcaattaaaagtcggcgacggtacggttattctgaatcaacaagccgatgccgacaaaaaagtccaggctttctcccaagtcggcattgtcagcggacgcggtacattggtattaaatagttcaaatcagattaatcccgataacctatatttcggtttccgtggcggtcgtttggatgccaatggcaatgacttgacttttgaacacatccgcaacgtggatgaaggcgcgcgcattgtcaaccacaacacaggccacgcctccacaatcacgctaacgggtaaatctttgattaccgatcccaaaaccatctctattcattatattcaaaataatgatgatgacgatgctggttattactattaccgccctaggaaaccgattccacaaggcaaagatctttatttcaaaaactaccgttattacgccctaaaatccggcggcagcgtgaacgcaccgatgcccgaaaacggacaaacggaaaataacgactggattttaatgggcagcacacaagaagaagccaagaaaaacgcgatgaaccacaaaaacaatcagcgtattagcggttttagcggtttctttggcgaagaaaacggaaaaggacataacggtgcattaaaccttaatttcaacggcaaaagcgcgcaaaaccgtttcttgttaacaggcggcactaatttaaacggaaaaataagcgtaactcaaggcaatgtcttgttatcaggtcgtccaacaccacacgcaagagattttgtgaacaaatcttcagcccgaaaagacgcacatttctccaaaaacaatgaagtcgtatttgaagacgactggataaaccgcacattcaaagccacagaaattgcggttaatcaatccgcatcattctcttccggcagaaatgtatccgacatcaccgccaacatcaccgcaacagacaacgccaaagtaaatttgggttacaaaaacggcgatgaggtttgcgtgcgctcggactataccggttacgttacctgcaacacaggcaacttatccgataaggctttaaacagctttggagcgacacagattaacggcaatgtgaatctgaaccagaatgcggcattggtcttgggcaaggctgcattatgggggcaaattcaagggcaaggaaacagccgtgtcagcctaaaccaacatagcaaatggcatttgaccggcgacagccaagtacacaatctgtcattggcggatagccatattcatttgaacaatgcctccgatgcgcaaagtgcaaataaataccacacgctcaaaatcaatcatttatccggtaacgggcattttcattatctgacgcacctggcgaaaaatcttggggataaagtgcttgtgaaggaatccgcatccggtcattatcagctccatgttcaagataaaacaggcgaacccaatcaggaagggctgaatctctttgatgcatcatccgtacaagaccgctcccgcctttctgtttccttggcgaataaccatgtagatttaggggcattgcgttatacaatcaaaacagaaaacggtattacccgattgtacaatccttatgccgagaaccgccgccgagtcaaaccggccccgtctcctgccacaaacacggcttctcaagcacaaaaggcaacacaaacggacggtgcacaaattgccaagcctcaaaatatcgtcgtcgcaccgcctagcccgcaggcaaatcaagccgaagaagcaaaacgccagcaagcaaaagcggagcaagtgaagcgtcagcaagcagaagcagagcgtaaatcggcagagttagcaaaacaaaaagcagaagccgaacgcgaagcaagagagctggcgacccgccaaaaagcagaacaagaacgcagcagcgccgaacttgcacgtcgacatgaaaaagaacgcgaagcggcagagttgtcggcaaaacaaaaagtcgaagccgaacgtgaggctcaagcattggcggtacgccgaaaagcagaagccgaagaggcaaaacgccaagctgcagagcttgcacgccggcatgaaaaagaacgcgaagcggcagagttgtcggcaaaacaaagggtcggggaagaggaacgccgtcaaacagcccaatcccagccgcaacgccgcaaacgccgtgccgcaccgcaggattatatggcagcttcccaagaccgtcctaaacgccgcggacaccgttcggtacagcaaaacaatgtggaaattgcccaagcccaagcagaacttgcgcgccgacagcaggaagaacgcaaagcggcagagttgttggcaaaacaaagggcagaagccgaacgtgaagctcaagcattggctgcgcgccgaaaagcagaagccgaagaggcaaaacgccaagctgccgaacttgcacatcgccaggaagcagaacgcaaagcggcagaattgtcggcaaaccaaaaagccgcagctgaagcccaagcattggcggcacgccaacaaaaagcccttgcgcgtcaacaggaagaagcacgaaaagctgcagagttagccgtcaaacaaaaagcggaaaccgaacgcaaaaccgccgaacttgccaagcaaagggcagcagccgaagcagcaaaacgccagcaagaagctcgccagaccgccgaacttgcacgtcgtcaagaagcggaacgccaagcagcagagttgtcggcaaaacaaaaagccgaaacagatcgagaagcggcagaatcggcgaaacgaaaagctgaggaagaagagcaccgtcaagcagcccaatcccagccgcaacgccgaaaacgccgtgccgcacctcaggattatatggcagcttcccaaaatcgtccgaaacgtcgcggacgcagatctactctgccggcaccgccctcgccatcatttgattcgtcagcttacgcagcacccagggccttgcataatccggactggtatgaaaatgactatgaagaaatccccttggatgcgttggaagatgaaaatgtatctgaatcagttgacacatcagacaaacagcctcaagacaatacggaacttcatgaaaaatatgaaaatgattatgaagaaatccccttggatgcgttggaagatgaagatgtatccgaatcggttgacacatcagacaaacagcctcaagacaatacggaacttcatgaaaaagttgagacagtgagtttgcaaccaagagccgcgcagccgcgagcccaagccgccactcagctgcaagcccaagccgccgcgcaagccgatgcagtcagcaccaataccaactcggctttatctgacgcaatggcaagcacgcaatctatcttgttggatacaggtgcttcattaacacggcacattgcacaaaaatcacgcgctgatgccgaaaaaaacagtgtttggatgtcgaacaccggttatggtcgtgattatgcttccgcacaatatcgccggtttagttcgaaacgcacgcaaacacaaatcggcattgaccgcagcttgtccgaaaatatgcagataggcggcgtattgacttactctgacagtcagcatacttttgatcaggcgagcggcaaaaatacttttgtgcaagccaacctttatggtaagtattatttaaatgatgcttggtatgtggccggcgatattggtgcaggcagcttgagaagccggttacaaacgcagcaaaaagcaaactttaaccgagcaagcatccaaaccggccttactttgggcaatacgctgaaaatcaatcaattcgagattgtccccagtgcggggatccgttacagccgcctgtcatctgcagattacaagttgggtaacgacagtgttaaagtaagttctatgtcagtgaaaacactaacggccggactggattttgcttatcggtttaaagtcggcaaccttaccgtaaaacccttgttatctgcagcttactttgccaattatggcaaaggcggcgtgaatgtgggcggtaattccttcgtctataaagcagataatcaacagcaatattcagcaggtgccgcgttactgtaccgtaatgttacattaaacgtaaatggcagtattacaaaaggaaaacaattggaaaaacaaaaatccggacaaattaaaatacagattcgtttctaa

SEQ ID NO: 3 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 4 - DNA, 4374 bp, Neisseria meningitidis
  source 1..4374: /mol_type=“unassigned DNA” /organism=“Neisseria meningitidis”
atgaaaacaaccgacaaacggacaaccgaaacacaccgcaaagccccgaaaaccggccgcatccgcttctcgcctgcttacttagccatatgcctgtcgttcggcattcttccccaagcctgggcgggacacacttatttcggcatcaactaccaatactatcgcgactttgccgaaaataaaggcaagtttgcagtcggggcgaaagatattgaggtttacaacaaaaaaggggagttggtcggcaaatcaatgacaaaagccccgatgattgatttttctgtggtgtcgcgtaacggcgtggcggcattggtgggcgatcaatatattgtgagcgtggcacataacggcggctataacaacgttgattttggtgcggaaggaagaaatcccgatcaacatcgttttacttataaaattgtgaaacggaataattataaagcagggactaaaggccatccttatggcggcgattatcatatgccgcgtttgcataaatttgtcacagatgcagaacctgttgaaatgaccagttatatggatgggcggaaatatatcgatcaaaataattaccctgaccgtgttcgtattggggcaggcaggcaatattggcgatctgatgaagatgagcccaataaccgcgaaagttcatatcatattgcaagtgcgtattcttggctcgttggtggcaatacctttgcacaaaatggatcaggtggtggcacagtcaacttaggtagtgaaaaaattaaacatagcccatatggttttttaccaacaggaggctcatttggcgacagtggctcaccaatgtttatctatgatgcccaaaagcaaaagtggttaattaatggggtattgcaaacgggcaacccctatataggaaaaagcaatggcttccagctggttcgtaaagattggttctatgatgaaatctttgctggagatacccattcagtattctacgaaccacgtcaaaatgggaaatactcttttaacgacgataataatggcacaggaaaaatcaatgccaaacatgaacacaattctctgcctaatagattaaaaacacgaaccgttcaattgtttaatgtttctttatccgagacagcaagagaacctgtttatcatgctgcaggtggtgtcaacagttatcgacccagactgaataatggagaaaatatttcctttattgacgaaggaaaaggcgaattgatacttaccagcaacatcaatcaaggtgctggaggattatatttccaaggagattttacggtctcgcctgaaaataacgaaacttggcaaggcgcgggcgttcatatcagtgaagacagtaccgttacttggaaagtaaacggcgtggcaaacgaccgcctgtccaaaatcggcaaaggcacgctgcacgttcaagccaaaggggaaaaccaaggctcgatcagcgtgggcgacggtacagtcattttggatcagcaggcagacgataaaggcaaaaaacaagcctttagtgaaatcggcttggtcagcggcaggggtacggtgcaactgaatgccgataatcagttcaaccccgacaaactctatttcggctttcgcggcggacgtttggatttaaacgggcattcgctttcgttccaccgtattcaaaataccgatgaaggggcgatgattgtcaaccacaatcaagacaaagaatccaccgttaccattacaggcaataaagatattgctacaaccggcaataacaacagcttggatagcaaaaaagaaattgcctacaacggttggtttggcgagaaagatacgaccaaaacgaacgggcggctcaaccttgtttaccagcccgccgcagaagaccgcaccctgctgctttccggcggaacaaatttaaacggcaacatcacgcaaacaaacggcaaactgtttttcagcggcagaccaacaccgcacgcctacaatcatttaaacgaccattggtcgcaaaaagagggcattcctcgcggggaaatcgtgtgggacaacgactggatcaaccgcacatttaaagcggaaaacttccaaattaaaggcggacaggcggtggtttcccgcaatgttgccaaagtgaaaggcgattggcatttgagcaatcacgcccaagcagtttttggtgtcgcaccgcatcaaagccacacaatctgtacacgttcggactggacgggtctgacaaattgtgtcgaaaaaaccattaccgacgataaagtgattgcttcattgactaagaccgacatcagcggcaatgtcgatcttgccgatcacgctcatttaaatctcacagggcttgccacactcaacggcaatcttagtgcaaatggcgatacacgttatacagtcagccacaacgccacccaaaacggcaaccttagcctcgtgggcaatgcccaagcaacatttaatcaagccacattaaacggcaacacatcggcttcgggcaatgcttcatttaatctaagcgaccacgccgtacaaaacggcagtctgacgctttccggcaacgctaaggcaaacgtaagccattccgcactcaacggtaatgtctccctagccgataaggcagtattccattttgaaagcagccgctttaccggacaaatcagcggcggcaaggatacggcattacacttaaaagacagcgaatggacgctgccgtcaggcacggaattaggcaatttaaaccttgacaacgccaccattacactcaattccgcctatcgccacgatgcggcaggggcgcaaaccggcagtgcgacagatgcgccgcgccgccgttcgcgccgttcgcgccgttccctattatccgttacaccgccaacttcggtagaatcccgtttcaacacgctgacggtaaacggcaaattgaacggtcagggaacattccgctttatgtcggaactcttcggctaccgcagcgacaaattgaagctggcggaaagttccgaaggcacttacaccttggcggtcaacaataccggcaacgaacctgcaagcctcgaacaattgacggtagtggaaggaaaagacaacaaaccgctgtccgaaaaccttaatttcaccctgcaaaacgaacacgtcgatgccggcgcgtggcgttaccaactcatccgcaaagacggcgagttccgcctgcataatccggtcaaagaacaagagctttccgacaaactcggcaaggcagaagccaaaaaacaggcggaaaaagacaacgcgcaaagccttgacgcgctgattgcggccgggcgcgatgccgtcgaaaagacagaaagcgttgccgaaccggcccggcaggcaggcggggaaaatgtcggcattatgcaggcggaggaagagaaaaaacgggtgcaggcggataaagacaccgccttggcgaaacagcgcgaagcggaaacccggccggctaccaccgccttcccccgcgcccgccgcgcccgccgggatttgccgcaactgcaaccccaaccgcagccccaaccgcagcgcgacctgatcagccgttatgccaatagcggtttgagtgaattttccgccacgctcaacagcgttttcgccgtacaggacgaattagaccgcgtatttgccgaagaccgccgcaacgccgtttggacaagcggcatccgggacaccaaacactaccgttcgcaagatttccgcgcctaccgccaacaaaccgacctgcgccaaatcggtatgcagaaaaacctcggcagcgggcgcgtcggcatcctgttttcgcacaaccggaccgaaaacaccttcgacgacggcatcggcaactcggcacggcttgcccacggcgccgttttcgggcaatacggcatcgacaggttctacatcggcatcagcgcgggcgcgggttttagcagcggcagcctttcagacggcatcggaggcaaaatccgccgccgcgtgctgcattacggcattcaggcacgataccgcgccggtttcggcggattcggcatcgaaccgcacatcggcgcaacgcgctatttcgtccaaaaagcggattaccgctacgaaaacgtcaatatcgccacccccggccttgcattcaaccgctaccgcgcgggcattaaggcagattattcattcaaaccggcgcaacacatttccatcacgccttatttgagcctgtcctataccgatgccgcttcgggcaaagtccgaacacgcgtcaataccgccgtattggctcaggatttcggcaaaacccgcagtgcggaatggggcgtaaacgccgaaatcaaaggtttcacgctgtccctccacgctgccgccgccaaaggcccgcaactggaagcgcaacacagcgcgggcatcaaattaggctaccgctggtaa

SEQ ID NO: 5 - protein, 1431 aa, Neisseria meningitidis
MRFTHTTPFCSVLSTLGLFAVSPAYSSIVRNDVDYQYFRDFAENKGAFTVGASNISIQDKQGKILGRVLNGIPMPDFRVSNRQTAIATLVHPQYVNSVKHNVGYGSIQFGNDTQNPEEQAYTYRLVSRNPHPDYDYHLPRLNKLVTEISPTALSSVPLLGNGQPKANAYLDTDRFPYFVRLGSGTQQVRKADGTRTRTAPAYQYLTGGTPLKVLGFQNHGLLVGGSLTDQPLNTYAIAGDSGSPLFAFDKHENRWVLAGVLSTYAGFDNFFNKYIVTQPEFIRSTIRQYETRLDVGLTTNELIWRDNGNGNSTLQGLNERITLPIANPSLAPQNDSRHMPSEDAGKTLILSSRFDNKTLMLADNINQGAGALQFDSNFTVVGKNHTWQGAGVIVADGKRVFWQVSNPKGDRLSKLGAGTLIANGQGINQGDISIGEGTVVLAQKAASDGSKQAFNQVGITSGRGTAVLADSQQIKPENLYFGFRGGRLDLNGNNLAFTHIRHADGGAQIVNHNPDQAATLTLTGNPVLSPEHVEWVQWGNRPQGNAAVYEYINPHRNRRTDYFILKPGGNPREFFPLNMKNSTSWQFIGNNRQQAAEQVAQAENARPDLITFGGYLGENAQTGKAAPSYSKTNEAAIEKTRHIANAAVYGRPEYRYNGALNLHYRPKRTDSTLLLNGGMNLNGEVLIEGGNMIVSGRPVPHAYDHQAKREPVLENEWTDGSFKAARFTLRNHARLTAGRNTAHLDGDITAYDLSGIDLGFTQGKTPECYRSYHSGSTHCTPNAVLKAENYRALPATQVRGDITLNDRSELRLGKAHLYGSIRAGKDTAVRMEADSNWTLSQSSHTGALTLDGAQITLNPDFANNTHNNRFNTLTVNGTLDGFGTFRFLTGIVRKQNAPPLKLEGDSRGAFQIHVKNTGQEPQTTESLALVSLNPKHSHQARFTLQNGYADLGAYRYILRKNNNGYSLYNPLKEAELQIEATRAEHERNQQAYNQLQATDISRQVQHDSDATRQALQAWQNSQTELARIDSQVQYLSAQLKQTDPLTGILTRAQNLCAAQGYSADICRQVAKAADTNDLTLFETELDTYIERVEMAESELDKARQGGDAQAVETARHAYLNALNRLSRQIHSLKTGVAGIRMPNLAELISRSANTAVSEQAAYNTGRQQAGRRIDRHLTDPQQQNIWLETGTQQTDYHSGTHRPYQQTTNYAHIGIQTGITDRLSVGTILTDERTNNRFDEGVSARNRSNGAHLFVKGENGALFAAADLGYSNSRTRFTDYDGAAVRRHAWDAGINTGIKIDTGINLRPYAGIRINRSNGNRYVLDGAEINSPAQIQTTWHAGIRLDKTVELGQAKLTPAFSSDYYHTRQNSGSALSVNDRTLLQQAAHGTLHTLQIDAGYKGWNAKLHAAYGKDSNTARHKQAGIKIGYNW

SEQ ID NO: 6 - DNA, 4296 bp, Neisseria meningitidis
  source 1..4296: /mol_type=“unassigned DNA” /organism=“Neisseria meningitidis”
atgcgcttcacacacaccaccccattttgttccgtattgtccaccctcggtctttttgccgtttcccctgcttactcatccattgtccgcaacgatgtcgattaccaatattttcgcgactttgccgaaaataaaggcgcgttcaccgtaggtgcaagcaatatttccatccaagacaagcaaggcaaaatattaggcagggttctcaacggcatccccatgcccgacttccgcgtcagcaaccgccaaaccgccatcgccaccctggttcacccccaatacgtcaacagtgtcaaacacaacgtcggctacggttccatacaattcggcaacgacacccaaaatccagaagaacaagcctatacctaccgcctcgtatcacgcaacccgcacccggactacgactaccaccttccccgcctcaacaaactggttaccgaaatctcacctaccgcactcagcagcgtacccttgcttggaaacggccagccaaaggccaatgcctacctcgataccgaccgcttcccctactttgtacgactcggctcaggcacgcaacaagtccgcaaagcagacggcacgcgtacacgaaccgccccggcataccaatacctgaccggcggcacgccgctgaaagtattggggttccaaaaccacggcttactcgtcggcggcagcctgaccgaccaaccccttaacacctacgcaatcgccggagacagcggttcccccctgtttgccttcgacaagcatgaaaaccgctgggtgcttgcgggcgtactcagcacctacgccggcttcgataatttcttcaacaaatacatcgtcacgcaacccgaattcatccgttccaccatccgccaatacgaaacccggctggatgtcgggctgaccaccaacgaactcatatggcgcgacaacggtaatggcaacagcaccctgcaagggctcaacgaacgcatcaccctgcccattgcaaacccttcgcttgccccacaaaacgacagcaggcacatgccgtctgaagatgccggcaaaacgctcatcctatccagcaggttcgacaacaaaacactgatgctggcagacaatatcaaccaaggcgcaggcgcattgcagttcgacagcaacttcaccgtcgtcggtaaaaaccacacatggcaaggtgcaggcgttatcgtagccgacggcaaacgcgtcttctggcaagtcagcaaccccaaaggcgaccggctctccaaactgggcgcaggcacgcttatcgccaacggacaaggcatcaaccagggcgacatcagcatcggggaaggcactgtcgtactcgcccaaaaagctgcttcagacggcagcaaacaagcattcaaccaagtcggcatcaccagcggcaggggcacggccgtcctcgccgacagccagcaaatcaaacccgaaaacctctatttcggcttcaggggcggacggctcgacctcaacggcaacaaccttgcctttacccatatccgccatgcggacggcggcgcgcaaatcgtcaatcacaaccctgaccaagccgcgacactgacgctgaccggcaaccccgtcctcagtcccgagcatgtcgagtgggtgcaatggggcaaccgtccgcaaggcaacgcggcggtttacgaatacatcaacccgcaccgcaaccgtcggaccgactacttcatactcaaacccggcggcaacccgcgcgaatttttcccgttaaatatgaaaaactcaacaagctggcaatttatcggcaacaacaggcaacaggccgccgaacaagtcgcccaagccgaaaatgcccgccccgacctgattaccttcggcggatacttgggtgaaaacgcgcaaacgggcaaagccgcgccgagttacagcaaaaccaatgaagcagccatagaaaaaacccgccatatcgcaaatgccgccgtatacggccggcccgaataccgttacaacggcgcactcaacctgcactatcgtcccaaacgcaccgacagcacgctgttgctcaacggcggcatgaaccttaacggggaagtcttgattgagggcggcaatatgattgtgtcaggcaggcccgtaccccatgcctacgaccaccaggccaaacgcgaacccgttcttgaaaacgaatggaccgacggcagcttcaaggctgcacggttcaccctgcgaaaccatgcccgactgacggcagggcgcaataccgcgcatctggacggcgacataaccgcatacgatctgtccggcatcgacctcggctttacccaaggcaaaacaccggaatgctaccgctcctaccatagcggcagcacccactgcacacccaacgccgttttaaaagccgaaaactatcgtgcactacctgcaacgcaagtacgcggcgacattacccttaacgaccgttcagagctccgcctgggcaaagcacacctgtacggcagcatccgtgccggcaaagacaccgcagtccgcatggaagcagacagcaactggacactttcccagtccagccacaccggcgcactgacgcttgacggcgcacaaattaccctgaaccccgatttcgccaataatacacacaacaaccgcttcaacacactgaccgtcaacggcacacttgacgggttcggcacattccgattcctgaccggcatcgtccgaaaacaaaatgccccccccctcaaactggaaggggacagccgcggcgcattccaaatccacgtcaaaaacaccggacaagaacctcaaacaaccgaatcgcttgcacttgtgagcctcaatccgaaacacagccaccaagcccgattcaccctccaaaacggctatgccgatttgggtgcctaccgctacatcctccgcaaaaacaacaacggatacagcctgtacaacccgctcaaagaggccgaacttcaaattgaagccacgcgtgcggaacatgagcgcaaccaacaggcatacaaccaattacaggcaaccgacatcagcagacaggttcaacatgactctgacgcgaccaggcaggcactacaggcctggcagaacagtcaaaccgaacttgcccgcatcgacagccaagtccaatatctgtccgcccaattgaaacagacagacccgctgaccggcattctgacgcgtgcccaaaacctgtgtgccgcacaaggatacagtgccgatatctgccgtcaggttgccaaagccgccgacacgaacgacctgacactcttcgaaaccgaactggatacgtatatagaacgtgtagaaatggccgaatccgaacttgacaaagcacggcaaggcggcgatgcgcaagccgtcgaaacagcccggcacgcctacctgaacgcactcaaccgtctgtcccgacaaatccacagtttgaaaaccggcgttgccggcatccgtatgccgaacctggccgaactgatcagccggtcggccaacaccgccgtttccgaacaggccgcctacaataccggccggcaacaggcgggacgccgcatcgaccgccaccttaccgatccgcagcagcaaaacatctggctggaaaccggtacgcaacaaaccgactaccatagcggcacacaccgtccctaccaacaaactaccaactatgcacatatcggcatccaaaccggcatcaccgaccgtctcagtgtcggtacgattttaaccgatgagcgcacaaacaaccgttttgatgaaggcgtatccgcccgaaaccgcagcaacggcgcacatctgttcgtcaaaggggaaaacggcgcactctttgccgcggcagatttaggctacagcaacagccgtacccgatttaccgattatgacggggctgccgtccgccgccacgcatgggatgcaggcatcaacaccggcatcaaaatcgataccggcatcaacctcagaccctatgccggcatccgtataaaccgcagcaacggcaaccggtacgtactcgacggcgcagagataaacagcccggcgcaaatccaaaccacatggcatgccggcatccgtctcgataaaaccgtcgaactgggtcaagccaagctgacccccgccttcagcagcgattactaccatacccgccaaaacagcggttccgccctcagcgtcaacgaccgtaccttactgcagcaagccgcccacggcacactgcataccctgcaaatcgacgccggatacaaaggctggaacgccaaacttcatgccgcttacggcaaagacagcaacaccgcccgccacaaacaggcaggaatcaaaataggctacaactggtaa

(19) United States
(12) Patent Application Publication
(10) Pub. No.: US 2018/0125960 A1
(43) Pub. Date: May 10, 2018

(54) MENINGITIDIS VACCINES COMPRISING SUBTILINASES

(71) Applicant: SANOFI PASTEUR, Lyon (FR)

(72) Inventors: Nadège ARNAUD-BARBE, Saint Sorlin (FR); Geneviève RENAULD-MONGENIE, Chaponost (FR); Bachra ROKBI, Lyon (FR)

(21) Appl. No.: 15/534,441

(22) PCT Filed: Dec. 8, 2015
(86) PCT No.: PCT/EP2015/079001

§ 371 Date: Jun. 8, 2017

(30) Foreign Application Priority Data
Dec. 9, 2014 (EP) 14306976.3

(51) Int. Cl. A61K 39/095; C07K 14/22; A61K 39/00

(52) U.S. Cl. CPC A61K 39/095; C07K 14/22; C07K 2319/40; C07K 2319/21; C07K 2319/10; C07K 2319/50; A61K 2039/575

(57) ABSTRACT

The present invention relates to novel polypeptides derived from meningitis proteins, in particular auto-transporters of the subtilinase subclass, and their use in vaccines and vaccine compositions for the prevention and/or treatment of meningitis and meningococcal infections.

The present invention relates to novel polypeptides derived from Neisseria meningitidis proteins, in particular auto-transporters of the subtilinase subclass, and their use in vaccines and vaccine compositions for the prevention and/or treatment of meningitis and meningococcal infections. In particular it provides fragments of NalP and polypeptides comprising or consisting of said fragments, which may be of use in immunogenic compositions, for example vaccine compositions.

### BACKGROUND

Neisseria meningitidis is one of the most important causes of bacterial meningitis and septicemia worldwide in both endemic and epidemic forms. The bacteria are classified into serogroups based on the structure of their capsular polysaccharides. Thirteen different serogroups have been identified but only five (A, B, C, W135 and Y) are responsible for the majority of infections, although epidemic meningitis due to meningococcal serogroup X is emerging in the Meningitis Belt of Africa. Two effective quadrivalent polysaccharide-protein conjugate vaccines have been developed and licensed against serogroups A, C, W135 and Y. In contrast to the other capsular polysaccharides, the group B polysaccharide is not an appropriate vaccinal antigen because of structural similarities with polysialic acid chains present in human cells. These properties of the serogroup B polysaccharide have impeded the development of a polysaccharide-based vaccine against group B Neisseria meningitidis (MenB) and led to the development of alternative vaccines.

During in vitro culture conditions and in vivo infection, N. meningitidis releases outer membrane blebs which contain lipooligosaccharide (LOS) and outer membrane proteins (OMPs). These blebs are known as outer membrane vesicles (OMVs). Meningococcal OMV vaccines have been developed and shown to be successful in controlling outbreaks of MenB disease when using OMVs produced from the outbreak strain. Several approaches have been carried out to increase the breadth of coverage of OMV vaccines. Despite these developments and the suggestion that a vaccine including six PorA and five FetA variants would potentially provide protection against the majority of global circulating pathogenic strains, the search for a vaccine candidate that is highly conserved and expressed by all disease causing meningococci has continued.

Recently, a vaccine against N. meningitidis has been licensed and commercialized under the trade name Bexsero®. This vaccine contains three recombinant N. meningitidis serogroup B proteins, namely NHBA (Neisseria Heparin Binding Antigen) fusion protein, NadA protein and fHbp fusion protein, together with outer membranes vesicles (OMVs) from N. meningitidis serogroup B.

Reference to N. meningitidis herein may be understood to refer to any serogroup, or may be understood to refer specifically to the B serogroup.

Meninges outer membrane proteins (OMPs) are now considered as the most promising vaccine candidates for broadly protecting against all serogroups. A particular class of OMPs is constituted by auto-transporters.

Auto-transporters are virulence factors produced by Gram-negative bacteria. Auto-transporters are modular proteins initially expressed as a precursor consisting of an N-terminal signal sequence and a C-terminal translocator domain separated by an N-terminal passenger domain that is secreted into the extracellular medium. The signal sequence directs the auto-transporter to the secretion machinery for transport across the internal membrane. The translocator domain mediates the transport of the passenger domain across the outer membrane. The term auto-transporter was coined because of the apparent absence of dedicated secretion machinery.

On the basis of their N- and C-terminus domains, auto-transporters can be divided into several categories. The classical auto-transporters, as typified by the IgA1 protease, contain a catalytic site in the N-terminal half of the passenger domain which often, although not always, is involved in the autocatalytic release of the passenger domain from the cell surface. The catalytic site is constituted by an amino acid triad comprising a serine residue. Accordingly, these auto-transporters are classified as serine proteases.

The very first N. meningitidis auto-transporter that was described was indeed the IgA1 protease. The amino acid sequence precursor of this protein was first described in Lomholt et al., Mol. Microb. (1995) 15 (3): 495. Since then, the N. meningitidis IgA1 protease has been extensively studied and characterized (Vitovski & Sayers, Infect. Immun. (2007) 75 (6): 2875; Ulsen & Tommassen, FEMS Microbiol. Rev. (2006) 30 (2): 292). The IgA1 protease was proposed for vaccine use in the early nineties (WO 90/11367).

After becoming publicly available, the genomes of N. meningitidis strains MC58 (serogroup B) (Tettelin et al., Science (March 2000) 287: 1809), Z2491 (serogroup A) and FAM18 (serogroup C) have been systematically searched for the presence of genes encoding auto-transporters. BLAST searches using known auto-transporters as search leads resulted in the identification of eight genes putatively encoding proteins with auto-transporter characteristics, four of which encode serine proteases:
iga encoding IgA1 protease (IgA1P): NMB 0700, NMA 0905 and NMC 0651, respectively in the MC58 (serogroup B), Z2491 (serogroup A) and FAM18 (serogroup C) strain genomes;
app encoding App (adhesion and penetration protein): NMB 1985, NMA 0457 and NMC 1969, respectively in the MC58, Z2491 and FAM18 strain genomes;
ausI (mspA) encoding AusI (MspA): NMB 1998 in the MC58 genome; and
nalP (aspA) encoding NalP (AspA): NMB 1969, NMA 0478 and NMC 1943, respectively in the MC58, Z2491 and FAM18 strain genomes.

WO 00/26375 relates to several virulence factors of N. meningitidis for use as a vaccinal agent. One of these factors is the outer membrane protein ORF047 of N. meningitidis strain ATCC13090 of serogroup B, also designated under the reference NMB1969 (Tettelin et al., Science (March 2000) 287: 1809). This protein has been further identified as an auto-transporter lipoprotein with subtilisin-like serine protease activity, by Turner et al., Infect. Immun. (2002) 70: 4447 and originally called AusP or AspA for “auto-transported serine protease A”. Later on, in van Ulsen et al., Mol. Microbiol. (2003) 50 (3): 1017, this protein was called NalP for “N. meningitidis auto-transporter lipoprotease”. The nalP gene has been shown to be subject to phase variation.

Although the terms “ORF047”, “NMB1969”, “AspA” and “NalP” may be used interchangeably, the term “NalP” has been selected for further use hereinafter.

Like all auto-transporters, NalP is produced as a precursor of about 112 kDa, comprising a cleavable signal peptide, a N-terminal passenger domain and a C-terminal, outer-membrane-based beta-domain, this latter comprising in sequence, a N-terminal translocator domain, an alpha-domain helix and a C-terminal beta-core composed of 12 beta-sheets. The precursor is transported to the outer membrane and the C-terminal domain remains surface-exposed while the N-ter passenger domain of about 70 kDa is processed and secreted (released into the bacterial environment) upon auto-cleavage due to the subtilisin-like serine protease activity of NalP. van Ulsen et al, Mol. Microbiol. (2003) 50 (3): 1017 has additionally shown that the lipidated form is an intermediate in the secretion process, as the secreted 70 kDa form is not lipidated.

The domain structure of NalP is further described in Table 1 below by reference to NalP of strain MC58: SEQ ID NO: 1; numbering starts in amino acid position 1 with the Met initiation codon, followed by the peptide leader sequence; the full-length mature protein being considered to start with the Cysteine residue in position 28. The N-terminal passenger domain of NalP is responsible for the subtilisin-like serine protease activity. The serine protease catalytic site is constituted by a triad [Asp/His/Ser respectively at positions 138, 157 and 426 in NalP of strain MC58]. The twelve beta-sheets in the form of a barrel constitute a hydrophilic pore filled by the alpha-domain in the form of an alpha-helix.

TABLE 1
Domain structure of N. meningitidis MC58 NalP (NMB1969)

| | | NalP N-terminal domain = | NalP C-terminal domain = β domain | | |
|---|---|---|---|---|---|
| Catalytic | Signal | Passenger domain = | Translocator | | β core = |
| triad | sequence | Protease domain | domain | α-peptide | 12 β-sheets |
| 138D | 1-27 | 28-774 | 775-784 | 785-809 | 810-1082 |
| 157H | | | | | |
| 426S | | | | | |

It shall be understood that the domain definitions by reference to amino acid positions may vary slightly as it is not always possible to precisely define a domain to the exact amino acid. For example, the domains may be defined slightly differently by different workers or they may be defined differently in different strains of N. meningitidis. Thus, said domains may be defined according to the locations given herein and/or in the figures, or according to said locations +/−1, 2, 3, 4, 5, 6, 7, 8, 9, 10 or more amino acids, i.e. 1, 2, 3, 4, 5, 6, 7, 8, 9, 10 or more amino acids N-terminal and/or C-terminal of said locations.

The same holds true for the location of the twelve beta-sheets of strain MC58 beta-core; however it is indicated that they are located approximately as follows:

1st β-sheet L817-E831; 2nd β-sheet E836-G853; 3rd β-sheet T856-E871; 4th β-sheet A874-A891; 5th β-sheet G895-S913; 6th β-sheet H919-V937; 7th β-sheet D946-Q960; 8th β-sheet L977-P993; 9th β-sheet A998-D1009; 10th β-sheet T1039-G1052; 11th β-sheet W1055-S1066; 12th β-sheet Y1069-F1082.

The sequences of the nalP gene (ORF047) and NalP protein thereof were initially described in patent application WO 00/26375 (Sanofi Pasteur/INSERM). Full-length sequences were identified by analysis of the genome sequence of N. meningitidis serogroup B strain ATCC 13090, available in the database Pathoseq® of Incyte Pharmaceuticals.

nalP gene and NalP protein sequences of N. meningitidis strains MC58 (serogroup B), Z2491 (serogroup A) and FAM18 (serogroup C) are respectively disclosed in Tettelin et al., Science, March 2000, 287: 1809 or WO 00/66791; Parkhill et al., Nature (March 2000) 404: 502; and Bentley et al., PLoS Genet., 3, e23 (2007). MC58 NalP is also designated as NMB1969 and sequences thereof are available under accession number NP_003112 (version NP as submitted on Mar. 17, 2000). Z2491 NalP is also designated as NMA0478. FAM18 NalP is also designated as NMC1943.

### DESCRIPTION OF THE INVENTION

Auto-transporter proteins have been used as vaccine antigens against other bacterial species.

Surprisingly, it has now been found that the vaccine potential of NalP is improved when the passenger domain is unable to be auto-cleaved.

One aspect of the invention thus relates to a composition comprising a polypeptide comprising a full-length mature N. meningitidis NalP protein; wherein said full-length mature N. meningitidis NalP protein lacks or has reduced subtilisin-like serine protease activity and/or does not contain any cleavage site susceptible/able to be cleaved by a subtilisin-like serine protease.

In one embodiment, said polypeptide comprises a full-length mature NalP protein which is mutated so that it lacks or has reduced subtilisin-like serine protease activity and/or does not contain any cleavage site susceptible/able to be cleaved by a subtilisin-like serine protease.

Further, as NalP is a protein with low solubility due to the presence of the beta-core, recombinant expression and purification is difficult to achieve. In an attempt to overcome this problem, soluble truncated forms lacking the entire beta-domain were assayed for bactericidal activity. Negative results generated a second set of constructs lacking the beta-core exclusively, leading to unstable recombinant products. It was eventually found that stable NalP fragments with improved solubility should contain a partial beta-core. These fragment constructs suitable for recombinant expression also have improved vaccine potential when the passenger domain cannot be auto-cleaved.

Accordingly, the invention also relates to a NalP polypeptide comprising or consisting of:

(A) a fragment of a full-length mature N. meningitidis NalP protein (NalP fragment), which full-length mature N. meningitidis NalP protein comprises (a) a passenger domain comprising a subtilisin-like serine protease triad (Asp/His/Ser) and (b) a beta-domain comprising a translocator domain, an alpha-peptide and a beta-core composed of twelve beta-sheets; said NalP fragment comprising:

(i) at the N-terminus, the NalP passenger domain;

(ii) at the C-terminus, the beta-domain comprising the translocator domain, the alpha-peptide and at least one and no more than eleven NalP beta-sheets; or

(B) a mutant of said NalP fragment (A) which lacks or has reduced subtilisin-like serine protease activity and/or does not contain any cleavage site able to be cleaved by a subtilisin-like serine protease;

wherein said polypeptide does not comprise a full-length mature N. meningitidis NalP protein.

A full-length mature N. meningitidis NalP protein considered the invention may be as described above.

Advantageously, the NalP polypeptide is the NalP fragment or the mutant of the NalP fragment, as described.

According to another embodiment, the invention relates to a composition comprising a polypeptide as above-described or a polypeptide comprising a full-length mature N. meningitidis NalP protein, together with a pharmaceutically acceptable diluent or carrier; wherein said full-length mature N. meningitidis NalP protein lacks subtilisin-like serine protease activity or does not contain any cleavage site able to be cleaved by a subtilisin-like serine protease.

According to another embodiment, the invention relates to a nucleic acid encoding a polypeptide comprising or consisting of a fragment of a full-length mature N. meningitidis NalP protein or a mutant of said NalP fragment, as above-described.

According to another embodiment, the invention relates to a vector comprising a nucleic acid as above-described.

According to another embodiment, the invention relates to a host cell comprising a nucleic acid as above-described.

According to another embodiment, the invention relates to a polypeptide comprising or consisting of a fragment of a full-length mature N. meningitidis NalP protein or a mutant of said NalP fragment as above-described, or a composition as above-described, for use as a vaccine, and more preferably for use for the prevention and/or treatment of N. meningitidis B infection.

According to another embodiment, the invention relates to a vaccine composition comprising a polypeptide comprising or consisting of a fragment of a full-length mature N. meningitidis NalP protein or a mutant of said NalP fragment as above-described, or a composition as above-described.

By “full-length mature NalP” is meant the NalP protein lacking the NalP signal peptide.

Accordingly, by “full-length mature NalP” is meant the full-length mature NalP comprising (having) (i) a naturally-occurring full-length mature amino acid sequence or (ii) a naturally-occurring full-length mature amino acid sequence lacking at most the first 50 N-terminus amino acids and/or at most the last 20 C-terminus amino acids or (iii) a naturally-occurring full-length mature amino acid sequence fused to the 1, 2 or 3 amino acids of the C-terminus of the signal sequence.

Advantageously, the full-length mature NalP that is referred to, is not lipidated. To that end, the full-length mature NalP may lack at least the cysteine residue in position 1 of the mature form (position 28, by reference to SEQ ID NO: 1).

The term “fragment” of a reference sequence refers to a chain of contiguous nucleotides or amino acids that is shorter than the reference sequence.

Although the mutated NalP for use in the composition of the invention and polypeptides of the present invention are more particularly exemplified herein by reference to the amino acid sequence of MC58 NalP protein (that naturally-occurs in N. meningitidis strain MC58), polypeptides of any naturally-occurring/allelic variant of N. meningitidis strain M982/B16B6/MC58 are also encompassed within the scope invention as well as any variant that may result from genetic engineering. By extension, the term variant is therefore applied to amino acid sequences, proteins or fragments thereof other than MC58 sequences, proteins or fragments thereof. Variations in amino acid sequence may be introduced by substitution, deletion or insertion of one or more codons into the nucleic acid sequence encoding the protein that results in a change in the amino acid sequence of the protein without substantially affecting the tri-dimensional structure and/or the biological and/or immunogenic properties. Typically, the variation may result for an amino acid substitution that may be conservative or non-conservative, preferably conservative. A conservative substitution is an amino acid substitution in which an amino acid is substituted for another amino acid with similar structural and/or chemical properties.

In what follows, variants and/or mutants are described by reference to the amino acid sequence of reference (SEQ ID NO: 1). Such a description by reference is based on the prerequisite of optimal sequence alignment in order to determine the amino acid in the variant sequence that corresponds to the amino acid defined as being in a specific position in the amino acid of reference.

In what follows, variants and/or mutants are also described by percent identity with a sequence of reference. Percent identity between two amino acid sequences or two nucleotide sequences is determined with standard alignment algorithms as those described below.

Sequence alignment can be achieved and percent identity can also be determined by standard local alignment algorithms such as the Smith-Waterman algorithm (Smith et al., J. Mol. Biol. (1981) 147: 195) (available on the EBI web site) or Basic Local Alignment Tools (BLASTs, including BLASTP for amino acid sequence alignment and BLASTN for nucleotide sequence alignment; described in Altschul et al., (1990) J. Mol. Biol., 215: 403) available on the National Center for the Biotechnology Information (NCBI) web site at http://www.ncbi.nlm.nih.gov/BLAST and may be used using the default parameters [BLASTP: Expect value E: 10; Word size: 3; Matrix: BLOSUM62; Cost gap: Existence 11, Extension 1; and BLASTN: algorithm by default: Megablast; Expect value E: 10; Word size: 28; Match scores: 1; Mismatch score: −2; Gap costs: linear (determined by the match/mismatch score) (October 2013)].

In the context of the invention, variant and mutant amino acid sequences include amino acid sequences that have at least about 80% sequence identity with an amino acid sequence defined herein. Preferably, a variant or mutant amino acid sequence will have at least about 80%, 81%, 82%, 83%, 84%, 85%, 86%, 87%, 88%, 89%, 90%, 91%, 92%, 93%, 94%, 95%, 96%, 97%, 98%, 99% sequence identity to a polypeptide sequence as defined herein. Amino acid sequence identity is defined as the percentage of amino acid residues in the variant sequence that are identical with the amino acid residues in the reference sequence, after aligning the sequences and if necessary, introducing gaps, to achieve the maximum percent sequence identity, and not considering any conservative substitution as part of the sequence identity. Standard alignment algorithms cited above are useful in this regard.

Variant and mutant nucleic acid sequences may include nucleic acid sequences that have at least about 80% nucleic acid sequence identity with a nucleic acid sequence disclosed herein. Preferably, a variant or mutant nucleic acid sequence will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% nucleic acid sequence identity to a full-length nucleic acid sequence or a fragment of a nucleic acid sequence as described herein. Nucleic acid sequence identity is defined as the percentage of nucleic acids in the variant sequence that are identical with the nucleic acids in the reference sequence, after aligning the sequences and introducing gaps, if necessary, to achieve the maximum percent sequence identity, and not considering any conservative substitutions as part of the sequence identity. Standard alignment algorithms cited above are useful in this regard.

An example of a full-length mature NalP protein is the full-length mature NalP protein of strain MC58. This MC58 NalP protein comprises (has) an amino acid sequence shown in SEQ ID NO: 1, starting with the amino acid in position 28, 29 or 30, preferably 29 or 30, and ending with the amino acid in position 1082. Other MC58 NalP proteins comprise (have) an amino acid sequence shown in SEQ ID NO: 1, starting with the amino acid in any one of the positions 28 to 78, preferably 28 to 58, more preferably 28 to 38, still more preferably in position 29 or 30, and ending with the amino acid in any one of the positions 1070 to 1082.

Other full-length mature NalP proteins include variants of MC58 NalP protein which may be described by reference to the MC58 amino acid sequence reported in SEQ ID NO: 1. These variants comprise (have) an amino acid sequence starting with an amino acid corresponding to the amino acid in position 28, 29 or 30, more preferably 29 or 30, and ending with an amino acid corresponding to the amino acid in position 1082. Other variants of the MC58 NalP protein, still described by reference to the MC58 amino acid sequence reported in SEQ ID NO: 1, comprise (have) an amino acid sequence starting with an amino acid corresponding to the amino acid in any one of the positions 28 to 78, preferably 28 to 58, more preferably 28 to 38, and ending with an amino acid corresponding to the amino acid in any one of positions 1070 to 1082.

According to a preferred embodiment, an isolated polypeptide according to the invention may comprise

(i) a passenger domain consists of residues at positions 28-774+/−2 amino acids on the C-terminal and/or N-terminal end of said locations of the amino acid sequence of SEQ ID NO:1; and/or

(ii) a beta domain consists of residues at positions 810-1082+/−2 amino acids on the C-terminal and/or N-terminal end of said locations of the amino acid sequence of SEQ ID NO: 1.

A full-length mature NalP protein for use in a composition of the invention lacks or has reduced subtilisin-like serine protease activity and/or does not contain any cleavage site susceptible/able to be cleaved by a subtilisin-like serine protease i.a., NalP.

An NalP passenger domain at the N-terminus of the NalP fragment may be the MC58 NalP passenger domain comprising (having) an amino acid sequence starting in position 28, 29 or 30 or in any one of position 28 to 78, preferably 28 to 58, more preferably 28 to 38, and ending in position 774 in SEQ ID NO: 1. It may also be a variant of the MC58 NalP passenger domain comprising (having) an amino acid sequence described by reference to the MC58 amino acid sequence reported in SEQ ID NO: 1, starting with an amino acid corresponding to the amino acid in position 28, 29 or 30 or in any one of the positions 28 to 78, preferably 28 to 58, more preferably 28 to 38, and ending with an amino acid corresponding to the amino acid in position 774. Preferably, an NalP passenger domain described above may start at position 29.

Advantageously, an NalP fragment comprises at the C-terminus at least one and no more than eight, six, four, or preferably two NalP beta sheets. In some embodiments, a NalP fragment comprises (i) the first beta-sheet; (ii) first and second beta-sheets; (iii) first, second and third beta-sheets; (iv) first, second, third and fourth beta-sheets; (v) first, second, third, fourth and fifth beta-sheets; (vi) first, second, third, fourth, fifth and sixth beta-sheets; (viii) first, second, third, fourth, fifth, sixth and seventh beta-sheets; or (viii) first, second, third, fourth, fifth, sixth, seventh, and eighth beta-sheets.

As a matter of example, a useful NalP fragment is the MC58 NalP fragment comprising at the C-terminus, two beta-sheets comprising (having) an amino acid sequence starting in any one of positions 815 to 820, e.g. in position 817 and ending in any one of positions 850 to 855, e.g. in position 853. Still as a matter of example, a useful NalP fragment is a variant of the MC58 NalP fragment comprising two beta-sheets comprising (having) an amino acid sequence, described by reference to the MC58 amino acid sequence reported in SEQ ID NO: 1, starting with an amino acid corresponding to the amino acid in any one of the positions 815 to 820, e.g. in position 817, and ending with an amino acid corresponding to the amino acid in any one of positions 850 to 855, e.g. in position 853.

For use in the present invention, an NalP fragment advantageously comprises a truncated NalP C-terminal domain including the translocator domain, the alpha-peptide and as described above, at least one and no more than eleven NalP beta-sheets.

An example of a useful NalP fragment is a fragment of the full-length mature NalP protein of strain MC58. Accordingly, this MC58 NalP fragment comprises (has) the amino acid sequence shown in SEQ ID NO: 1, starting with the amino acid in position 28, 29 or 30 and ending with the amino acid in position 853. Other useful MC58 NalP fragments comprise (have) the amino acid sequence shown in SEQ ID NO: 1, starting with the amino acid in position 28, 29 or 30 or in any one of the positions 28 to 78, preferably 28 to 58, more preferably 28 to 38, and ending with the amino acid in any one of positions 840 to 860, preferably 850 to 855. Preferably, an NalP fragment described above may start at position 29.

Other useful NalP fragments include variants of MC58 NalP fragments which may be described by reference to the MC58 amino acid sequence reported in SEQ ID NO: 1. These variants comprise (have) an amino acid sequence starting with an amino acid corresponding to the amino acid in position 28, 29 or 30 and ending with an amino acid corresponding to the amino acid in position 853. Other useful variants of the MC58 NalP fragment, still described by reference to the MC58 amino acid sequence reported in SEQ ID NO: 1, comprise (have) an amino acid sequence starting with an amino acid corresponding to the amino acid in position 28, 29 or 30 or in any one of the positions 28 to 78, preferably 28 to 58, more preferably 28 to 38, and ending with an amino acid corresponding to the amino acid in any one of positions 840 to 860, preferably 850 to 855. Preferably, an NalP fragment described above may start at position 29.

As already mentioned above, in some embodiments, a full-length mature NalP protein or an NalP fragment is mutated so that it lacks or has reduced subtilisin-like serine protease activity and/or does not contain any cleavage site susceptible/able to be cleaved by a subtilisin-like serine protease i.a., NalP. As a result of the mutation, NalP remains in a precursor state, the N-terminal passenger domain not being cleaved from the C-terminal domain. For example, protease activity may be reduced by 10, 20, 30, 40, 50, 60, 70, 80, 90, 95, 96, 97, 98, 99 or 100% compared to the wild type sequence. Protease activity may be evaluated by assaying the ability to cleave auto-transporter proteins, for example by Western Blot, as described in for example Roussel-Jazédé et al., Infect. Immun. (2010) 78 (7): 3083; van Ulsen P. et al., Mol. Microbiol. (November 2003) (3): 1017 and Serruto et al., PNAS Feb. 2010 107 (8): 3770.

Preferably, a full-length mature NalP protein or a NalP fragment lacks subtilisin-like serine protease activity. In order to reduce or abolish the serine protease activity, any of the amino acids present in the catalytic triad may be mutated, advantageously by amino acid substitution. In a particular embodiment, one way to achieve that goal may be to substitute the Serine residue in the catalytic triad by any other amino acid, advantageously by Glycine, Threonine, Valine, Leucine, Isoleucine or Alanine, preferably by Valine or Alanine, and more preferably by Alanine.

The catalytic triad of MC58 NalP is composed of Asp138, His157 and Ser426 in SEQ ID NO: 1. Accordingly, the catalytic triad of variant of MC58 NalP is composed of amino acids corresponding to Asp138, His157 and Ser426 in the amino acid sequence of SEQ ID NO: 1; and accordingly, the mutation occurs at the position corresponding to Asp138, His157 or Ser426 of the amino acid sequence of SEQ ID NO: 1.

Examples of mutated full-length mature NalP protein for use in the composition of the invention include in particular any one of the MC58 NalP full-length mature NalP protein or variants thereof as described above, each being mutated so that it lacks or has reduced subtilisin-like serine protease activity and/or does not contain any cleavage site susceptible/able to be cleaved by a subtilisin-like serine protease.

Examples of useful mutated NalP fragment include in particular any one of the MC58 NalP fragments or variants thereof as described above, each being mutated so that it lacks or has reduced subtilisin-like serine protease activity and/or does not contain any cleavage site susceptible/able to be cleaved by a subtilisin-like serine protease.

According to a preferred embodiment, an isolated polypeptide according to the invention may be a polypeptide lacking protease activity, and wherein the NalP passenger domain portion of said polypeptide has an amino acid substitution at the position corresponding to Asp138, His157 or Ser426 of the amino acid sequence of SEQ ID NO:1.

According to a preferred embodiment, an isolated polypeptide according to the invention may be a polypeptide wherein the residue corresponding to Ser426 of SEQ ID NO: 1 is substituted by Ala.

According to a preferred embodiment, an isolated polypeptide according to the invention may be a NalP fragment comprising no more than two NalP beta sheets.

As a matter of non-limiting illustration, particular examples include:
The S426A mutated MC58 NalP protein which comprises (has) the amino acid sequence shown in SEQ ID NO: 1, starting with the amino acid in position 29 or 30 and ending with the amino acid in position 1082; and in which Serine 426 is substituted by Alanine.
A mutated variant of the S426A MC58 NalP protein which may be described as follows by reference to the MC58 amino acid sequence reported in SEQ ID NO: 1. This variant comprises (has) an amino acid sequence starting with an amino acid corresponding to the amino acid in position 29 or 30 and ending with an amino acid corresponding to the amino acid in position 1082 and in which the amino acid corresponding to the Serine 426 is substituted by Alanine.
The S426A mutated MC58 NalP fragment which comprises (has) the amino acid sequence shown in SEQ ID NO: 1, starting with the amino acid in position 29 or 30 and ending with the amino acid in position 853; and in which Serine 426 is substituted by Alanine; and,
A mutated variant of the S426A MC58 NalP fragment which may be described as follows by reference to the MC58 amino acid sequence reported in SEQ ID NO: 1. This variant comprises (has) an amino acid sequence starting with an amino acid corresponding to the amino acid in position 29 or 30 and ending with an amino acid corresponding to the amino acid in position 853 and in which the amino acid corresponding to the Serine 426 is substituted by Alanine.

In some embodiments, a polypeptide of the invention may comprise or consist of a NalP fragment which essentially consists of the passenger domain, the translocator domain, the alpha-peptide and the first and second beta-sheets; wherein the Ser residue of the catalytic triad in the NalP passenger domain is optionally mutated by substitution.

The invention also provides a composition comprising a polypeptide comprising a full-length mature N. meningitidis NalP protein, together with a pharmaceutically acceptable diluent or carrier; wherein said full-length mature N. meningitidis NalP protein lacks or has reduced subtilisin-like serine protease activity and/or does not contain any cleavage site susceptible/able to be cleaved by a subtilisin-like serine protease and has from at least 80% to less than 100% identity or at least 80%, 85%, 90%, 95%, 96%, 97%, 98% or 99% but not 100% identity with the MC58 amino acid sequence reported in SEQ ID NO: 1, starting with the amino acid in position 28, or preferably at position 29, or preferably at position 30, and ending with amino acid in position 1082 (MC58 full-length NalP). Said NalP polypeptide may comprise or consist of a sequence of amino acids G30-F1082 of SEQ ID NO: 1 comprising a Ser426Ala substitution.

The invention also provides an isolated polypeptide comprising or consisting of a fragment of full-length mature NalP protein of N. meningitidis MC58 or of a NalP variant having at least 80%, 85%, 90%, 95%, 96%, 97%, 98%, 99% or 100% identity with a MC58 amino acid sequence reported in SEQ ID NO: 1, starting with the amino acid in position 28, 29 or 30 and ending with amino acid in position 853. Said NalP polypeptide may comprise or consist of the sequence; amino acids G30-G853 of SEQ ID NO: 1; or amino acids G30-G853 of SEQ ID NO:1 comprising a Ser426Ala substitution (SEQ ID NO: 3).

According to a preferred embodiment, an isolated polypeptide according to the invention may be a fragment of full-length mature NalP protein of N. meningitidis MC58 or of a NalP variant having at least 95% identity with the NalP protein of N. meningitidis MC58.

In one embodiment, said NalP fragment comprises or consists in an amino acid sequence which has at least 95%, 96%, 97%, 98%, 99% or 100% amino acid sequence identity with the amino acid sequence of SEQ ID NO: 3.

In one embodiment, said NalP fragment comprises or consists in an amino acid sequence which has at least 95%, 96%, 97%, 98%, 99% or 100% amino acid sequence identity with the amino acid sequence of SEQ ID NO: 4.

In one embodiment, said NalP fragment comprises or consists in an amino acid sequence which has at least 95%, 96%, 97%, 98%, 99% or 100% amino acid sequence identity with the amino acid sequence of SEQ ID NO: 5.

In one embodiment, said NalP fragment comprises or consists in an amino acid sequence which has at least 95%, 96%, 97%, 98%, 99% or 100% amino acid sequence identity with the amino acid sequence of SEQ ID NO: 6.

According to a preferred embodiment, an isolated polypeptide according to the invention may be NalP fragment comprising or consisting in the amino acid sequence of SEQ ID NO: 3.

According to a preferred embodiment, an isolated polypeptide according to the invention may be NalP fragment comprising or consisting in the amino acid sequence of SEQ ID NO: 4.

According to a preferred embodiment, an isolated polypeptide according to the invention may be NalP fragment comprising or consisting in the amino acid sequence of SEQ ID NO: 5.

According to a preferred embodiment, an isolated polypeptide according to the invention may be NalP fragment comprising or consisting in the amino acid sequence of SEQ ID NO: 6.

All references to ‘comprising’ herein should also be understood as including ‘consisting of’.

Unless otherwise indicated, all the antigens/polypeptides/fragments/constructs/amino acid sequences are described throughout the specification from the N-terminus end to the C-terminus end. As a matter of example, a fragment described as consisting of the protease domain, the α-peptide domain and part of the beta-domain of the trypsin-like serine protease auto-transporter of N. meningitidis shall be understood as a fragment consisting of, from N-ter to C-ter, the protease domain, the α-peptide domain and part of the beta-domain, the C-ter of the protease domain being fused to the N-ter of the α-peptide domain, the C-ter of which being fused to the N-ter of ‘part of the beta-domain’. Fusion is conveniently achieved by covalent peptidic bound (amide linkage CO—NH).

The NalP polypeptides may be synthetized by any method well-known from the skilled person. Such methods include biological production methods by recombinant technology and means. In particular, nucleotide sequences encoding the N. meningitidis NalP and corresponding amino acid sequences thereof may be retrieved from a number of bioinformatics websites such as the site of the European Bioinformatics Institute or the National Center for Biotechnology Information (US). AS a matter of example, nalP and NalP sequences of strain MC58 may be retrieved from the Entrez Gene database of the NCBI (National Center for the Biotechnology Information) at http://www.ncbi.nlm.nih.qov under the accession number NC 003112.

Any desired encoding sequences may be conceived and designed by bioinformatics according to methods and software known in the art, such as the software pack Vector NTI of Invitrogen; chemically synthetized de novo; and finally cloned into expression vectors available in the art. Methods of purification that can be used are also well-known from the skilled person.

Accordingly, the invention also provides nucleic acids encoding the NalP fragments and polypeptides disclosed herein. Also, are provided vectors comprising said nucleic acids, e.g., DNA, for example expression vectors, and host cells comprising said nucleic acids and/or vectors.

Also, is provided a method of production of a polypeptide as described herein, the method comprising expressing said polypeptide from a vector as described herein. In particular a method of producing a polypeptide as described herein, comprises culturing a host cell e.g., a bacterial strain transformed with a vector (i) comprising a nucleotide sequence e.g., a DNA sequence, encoding a polypeptide as described herein and (ii) able to express said polypeptide.

Variant and mutant nucleic acid sequences e.g., DNA sequences, include sequences capable of specifically hybridizing to the nucleotides sequences of strain MC58 encoding a polypeptide described herein under moderate or high stringency conditions.

Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C.

Immunogenic Composition and Administration

The invention also provides a composition comprising:

(I) A polypeptide comprising a full-length mature N meningitidis NalP protein; wherein said full-length mature N meningitidis NalP protein lacks or has reduced subtilisin-like serine protease activity and/or does not contain any cleavage site susceptible/able to be cleaved by a subtilisin-like serine protease; or

(II) A polypeptide of the invention comprising or consisting of:

(A) a fragment of a full-length mature N meningitidis NalP protein (NalP fragment), which full-length mature N meningitidis NalP protein comprises (a) a passenger domain comprising a subtilisin-like serine protease triad (Asp/His/Ser) and (b) a beta-domain comprising a translocator domain, an alpha-peptide and a beta-core composed of twelve beta-sheets; said NalP fragment comprising:

(i) at the N-terminus, the NalP passenger domain;

(ii) at the C-terminus, the beta-domain including the translocator domain, the alpha-peptide and at least one and no more than eleven NalP beta-sheets; OR

(B) a mutant of said NalP fragment (A) which lacks or has reduced subtilisin-like serine protease activity and/or does not contain any cleavage site able to be cleaved by a subtilisin-like serine protease;

wherein said polypeptide does not comprise a full-length mature N. meningitidis NalP protein.

In what follows (I) and (II) are collectively referred to as “polypeptide of the invention”.

In a preferred embodiment, said composition is an immunogenic composition comprising i.a., an immunologically effective amount of a NalP protein mutated as described above or a polypeptide of the invention. In a preferred embodiment, said composition is a pharmaceutical e.g., vaccine composition, comprising i.a., a pharmaceutically, prophylactically and/or therapeutically effective amount of a NalP protein mutated as described above or a polypeptide of the invention, i.a., together with a pharmaceutically acceptable excipient, e.g. a diluent or carrier.

Adjuvants

In a particular embodiment of the invention, the composition according to the invention comprises one or several adjuvant(s).

The term “adjuvant” as used herein denotes a product which, added to the content of an immunogenic composition, in particular to a vaccine, increases the intensity of the immune reaction induced in the mammalian host to which said composition is administered. An adjuvant may in particular increase the quantity/quality of specific antibodies e.g. bactericidal antibodies, which said host is capable of producing after administration of said composition and thus increases the efficiency of the immune response.

The adjuvant(s) that can be used in the context of the invention include adjuvants promoting a Th1 and/or Th2 immune response. Accordingly, for use in the composition of the invention, an adjuvant may be a Th1, Th2 or Th1/Th2 adjuvant. The meaning given to “Th1, Th2 or Th1/Th2 adjuvant” shall be the meaning commonly acknowledged by the scientific community. A Th1 adjuvant promotes an immune response characterized by the predominant production of IFN-γ and/or IL-2 cytokines. A Th2 adjuvant promotes an immune response characterized by the predominant production of e.g., IL-4, IL-5, IL-6 and/or IL-10 cytokines. A Th1/Th2 adjuvant favours a balanced cytokine production (balanced immune response).

Examples of adjuvants promoting a Th1-type immune response include but are not limited to agonists of Toll-like receptors (TLRs), in particular agonists of TLR4, which may be formulated or not. Typical formulation of a TLR agonist such as a TLR4 agonist, include oil-in-water emulsions. LPS derivatives like 3-De-O-acylated Monophosphoryl Lipid A (3D-MPL) described in WO 94/00153 or a 3D-MPL derivative named RC-529 described in U.S. Pat. No. 6,113,918 are well known TLR4 agonists. Other TLR4 agonists which share structural similarity with monophosphoryl lipid A, referred to as aminoalkyl glucosaminide phosphates (AGPs), are described in U.S. Pat. No. 6,113,918, U.S. Pat. No. 6,303,347, and WO 98/50399. Other synthetic TLR4 agonists are described in US 2003/0153532. Among these synthetic agonists, reference is made as particularly suitable Th1-adjuvant in the context of the invention to a chemical compound named as E6020 and referenced in the Chemical Abstract Services (CAS) registry as CAS Number 287180-63-6. The chemical formula of the disodic salt is C83H63N4019P2, 2Na and the developed chemical formula is as follows:

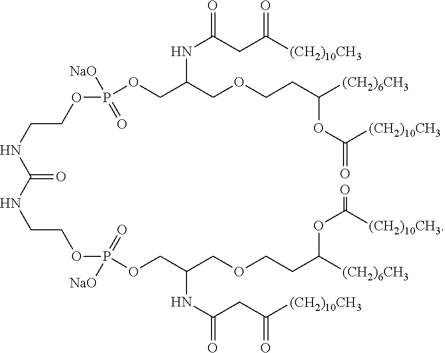

The R configuration (R,R,R,R) of the four asymetric carbons is preferred. The synthesis process is described in WO2007/005583. E6020 is preferably formulated in an oil-in-water emulsion and more particularly formulated in an oil-in-water emulsion (such as the one described in WO 07/006939), according to the process as described in the patent application WO 2007/080308.

Examples of adjuvants promoting a Th2-type immune response include but are not limited to aluminium salts and especially aluminium oxy hydroxide (also called for sake of brevity aluminium hydroxide) or aluminum hydroxy phosphate (also called for sake of brevity aluminum phosphate). When an aluminium salt is used, the protein antigens may advantageously be adsorbed onto the aluminium salt.

Excipients

In a composition of the invention, the active ingredients may be formulated together with a pharmaceutically-acceptable excipient such as a pharmaceutically acceptable diluent or carrier. In a particular embodiment, the composition of the invention may comprise a buffer and/or an isotonic agent such as sodium chloride or sugars e.g. sucrose; and/or a stabilizing agent such as histidine.

An immunogenic composition according to the invention is useful for inducing an immune response in a mammal, in particular humans, against N. meningitidis of any serogroup, in particular against serogroup B. This immune response includes in particular, a bactericidal immune response wherein bactericidal antibodies are induced against N. meningitidis. By “bactericidal antibody” is meant antibodies able to kill the bacteria in the presence of complement (which is a component of the humoral immune system of mammals). The antibodies produced as part of the immune response upon administration of the immunogenic composition may be identified as “bactericidal antibodies” in a serum bactericidal assay using an appropriate source of complement, according to methods known in the art.

Coverage of Protection

N. meningitidis species are genetically and antigenically highly diverse. Multilocus sequence typing (MLST) was first developed in the late 1990s for the meningococcus. It is a highly reliable and reproducible characterization method, which assesses variation at multiple genetic loci using nucleotide sequencing. More than 6751 sequence types (STs) have been assigned for N. meningitidis strains. While meningococcal diversity is extensive, it is highly structured. Studies of variation at housekeeping loci, initially by multilocus enzyme electrophoresis and more recently by MLST, had identified so far 37 groups of closely related meningococci, accounting for 61% of the meningococcal isolates represented in the PubMLST database. These groups, known as clonal complexes, have become the predominant unit of analysis in meningococcal population biology and epidemiology. A minority of clonal complexes, the so-called hyper-invasive lineages, are responsible for a disproportionate number of cases of disease worldwide and can be over-represented in collections of isolates from diseased patients by as much as two orders of magnitude, relative to their prevalence in asymptomatic carriage (see Table 2 herein after).

TABLE 2
Characteristics of the most important clonal complexes of Neisseria meningitidis
(data compiled from the PubMLST database Jun. 02, 2009).

| | | | | | | | Disease/ | |
|---|---|---|---|---|---|---|---|---|
| | | No. | No. | | Dominant | Dominant | carriage | |
| ST-complex | MLEE designation | isolates | STs | Dominant serogroups (%) | PorA | FetA | ratio | Main origin |
| ST-1 complex | Subgroup I/II | 204 | 49 | A (97) | 5-2, 10 | F3-5 | 5.5 | Russia, China |
| ST-5 complex | Subgroup III | 627 | 33 | A (99) | 20, 9 | F3-1 | 19.5 | Africa |
| ST-8 complex | Cluster A4 | 283 | 107 | B (51), C (35) | 5-1, 2-2 | F3-6 | 24.5 | Europe |
| ST-11 complex | ET-37 complex | 1142 | 239 | C (57), W135 (24), B(12) | 5, 2 | F3-6 | 6.6 | Worldwide |
| ST-18 complex | Cluster J1 | 208 | 175 | B (85) | 22, 14 | F3-6 | 5.5 | Czech Republic, Poland |
| ST-22 complex | | 363 | 243 | W135 (52), NG (25) | 18-1, 3 | F4-1 | 0.6 | UK |
| ST-23 complex | Cluster A3 | 385 | 154 | Y (62), NG (18) | 5-1, 2-2 | F4-1 | 0.8 | Worldwide |
| ST-32 complex | ET-5 complex | 1028 | 350 | B (85) | 19, 15 | F5-1 | 3.5 | Worldwide |
| ST-35 complex | | 329 | 214 | B (59), NG (25) | 22-1, 14 | F4-1 | 0.5 | Worldwide |
| ST-41/44 complex | Lineage 3 | 1796 | 1274 | B(70) | 7-2, 4 | F1-5 | 1.2 | Worldwide |
| ST-53 complex | | 272 | 93 | NG (76) | 7-2, 30 | F1-7 | <0.1 | UK |
| ST-60 complex | | 225 | 148 | B (30), 29E (22), NG (19) | 5, 2 | F1-7 | 0.7 | Europe |
| ST-103 complex | | 127 | 84 | B (26), NG (22), C (16) | 18-1, 3 | F3-9 | 1.2 | Worldwide (-Africa) |
| ST-162 complex | | 140 | 63 | B (74), NG (13) | 22, 14 | F5-9 | 0.8 | Worldwide |
| ST-167 complex | | 201 | 144 | Y (47), NG (36) | 5-1, 10-4 | F3-4 | 0.5 | Worldwide |
| ST-198 complex | | 166 | 76 | NG (76) | 18, 25-15 | F5-5 | <0.1 | Worldwide |
| ST-213 complex | | 187 | 165 | B (74), NG (16) | 22, 14 | F5-5 | 0.6 | UK |
| ST-254 complex | | 148 | 107 | NG (35), B (24), 29E (12) | 5-1, 16 | F1-7, F3-6 | 0.5 | Worldwide |
| ST-269 complex | | 415 | 312 | B (73) | 22, 9 | F5-1 | 2.8 | Worldwide |
| ST-334 complex | | 106 | 64 | C (58), B (33) | 5-1, 2-2 | F1-5 | 5.7 | UK |

As shown in the above table, strains of e.g., serogroup B, belong to several clonal complexes. In particular, serogroup B strains are highly represented among significant invasive clonal complexes, including major clonal complexes spread worldwide i.e., ST-8, ST-18, ST-32, ST-41/44, ST-162 and ST-269 clonal complexes, as well as clonal complex ST-11, remarkable for its very low rate of carriage relative to high incidence of disease.

It is therefore highly desirable to evaluate the protection coverage provided by a NalP mutated as described above or a polypeptide of the invention. To determine whether they are likely to give broad coverage across strains of serogroup B, representative strains of that serogroup among major clonal complexes (6 ST complexes or groups) were selected and effectiveness in term of protection coverage of these various protein antigens and combinations thereof was tested against these strains.

This protection coverage may be evaluated by serum bactericidal activity (SBA) assay which reflects the ability of a given antigen to elicit bactericidal antibodies. The SBA assay measures functional activity of antibody through complement-mediated antibody lysis of the bacteria. Serum bactericidal activity has been accepted as a valid surrogate for predicting the clinical efficacy of serogroup B meningococcal vaccines.

Indirect evidence of SBA assay providing surrogate of protection came from studies by Goldschneider and colleagues in 1969 where an inverse correlation between the incidence of disease and the prevalence of serum bactericidal activity in human serum SBA against MenA, MenB and MenC were reported. In their prospective study, the SBA titer in serum was measured using human complement (e.g., endogenous complement or exogenous serum from a healthy adult who lacked intrinsic bactericidal activity). They demonstrated in incoming recruits to a US Army base that the presence of serum bactericidal activity strongly indicated resistance to meningococcal disease. This led to the establishment of SBA assay as the immunological surrogate of protection against meningococcal disease.

Indeed, in clinical trials of MenB vaccines, the measurement of the increase of the SBA titer after vaccination compared to the SBA basal titer (before any administration of the meningococcal vaccine) is an established clinical end point. Seroconversion is considered to be met when an SBA titer is superior or equal to 4. This approach was validated in 2005 at a World Health Organization sponsored meningococcal serology standardisation workshop and is based upon evidence from a number of efficacy studies of OMV vaccines.

Animal SBA assays achieved in upstream research are as well commonly acknowledged as a surrogate of protection for N. meningitidis vaccines. In the context of the present invention, initial SBA assays were first carried out against the homologous strain (‘homologous’ assays). Then, antigens, in particular those that gave positive results in the homologous SBA assay were taken forward into ‘heterologous’ SBA assays, in which the antigen-specific corresponding sera were tested against different strains, to give an indication of the effectiveness of strain coverage which may be obtained. The inventors of the present invention have determined that when the SBA titer (fold-increase compared to a negative control group) is superior or equal to 16 in homologous SBA assay, or superior or equal to 8 in heterologous SBA, protection is considered to be met.

An optimal vaccine shall give a broad coverage against a panel of representative strains of relevant invasive clonal complexes.

Accordingly, an immunogenic composition according to the invention is particularly useful for inducing an immune response i.a., a bactericidal immune response, against N. meningitidis strains of (i) the clonal complexes of the hyper-invasive lineage (invasive clonal complexes); (ii) the clonal complexes wherein strains of serogroup B are prevalent (highly represented), those complexes being or not prevalent worldwide, advantageously prevalent worldwide; and/or (iii) clonal complexes ST8, ST11, ST18, ST32, ST41/44, ST-162 and/or ST269. The immunogenic composition is more particularly useful against N. meningitidis strains of serogroup B belonging to clonal complexes, such as the ST11, ST18, ST32 and/or ST41/44 complex(es). The immunogenic composition may be characterized by strain coverage of at least 50%. In other words, it may induce a bactericidal immune response against at least 70%, 75%, 80%, 85%, 90%, 95% or 100% of N. meningitidis strains in one of the clonal complexes specified above, in particular the ST8, ST11, ST18, ST32, ST41/44, ST-162 and/or ST269 complex(es).

Strain coverage may be determined as described in the experimental part of the specification, involving in particular (i) the selection of a collection of strains representative of the most important clonal complexes e.g. including ST8, ST11, ST18, ST32, ST41/44, ST-162 and/or ST269 complex(es) and (i) the achievement of an SBA assay against each of the strains of the collection, such as described in the experimental part. Briefly, the whole test consist in administering the composition to a mammal, one or several times at appropriate intervals; collecting the sera that may optionally be pooled (within a group of mammals submitted to identical administration); culturing the strains of the collection; and testing the individual sera or pooled serum and/or dilutions thereof against each strain in an SBA assay, such as the one described in the experimental part. The percentage of coverage is determined on the basis of the number of strains responding positively—that is, against which the bactericidal titer of e.g., the pooled serum, meets (e.g., equals or is superior to) the threshold value considered as indicative of a positive surrogate of protection—over the total number of strains tested. Alternatively, the bactericidal titer of individual sera within a group of mammals as defined above may be determined and the geometric mean titer (GMT) established. In that case, the strain is considered to respond positively when the GMT meets (e.g., equals or is superior to) the threshold value considered as indicative of a positive surrogate of protection.

An immunogenic composition according to the invention may be used as a pharmaceutical composition, in a prophylactic or therapeutic manner. Typically, it may be used as a vaccine composition for protecting against N. meningitidis infections e.g., for treating or preventing N. meningitidis infections. N. meningitidis induces a large range of infections from asymptomatic carriage to invasive diseases e.g., meningitis and/or septicemia. Typically, the immunogenic or pharmaceutical composition of the invention comprises a therapeutically or prophylactically effective amount of a polypeptide of the invention. A therapeutically and/or prophylactically effective amount of a polypeptide of the invention may vary according to factors such as the disease state, age, sex, and weight of the individual, and the ability of the polypeptide of the invention to elicit a desired therapeutic and/or prophylactic result.

The composition according to the invention may be administered as a dose wherein the amount of a polypeptide of the invention depends on various conditions including e.g., the weight, the age and the immune status of the recipient. As a matter of guidance, it is indicated that a dose of the composition of the invention may comprise a therapeutically and/or prophylactically effective amount of a polypeptide of the invention, which may be from 10 μg to 1 mg, e.g. about 50 μg.

‘Prevention’ refers to prophylactic treatment, wherein a composition of the invention is administered to an individual with no symptoms of meningitis and/or septicemia and/or no detectable N. meningitidis infection. Said prophylactic treatment is preferably administered with the aim of preventing or reducing future N. meningitidis infection.

Within the meaning of the invention, the terms “for preventing or for prevention” intend to mean, with reference to an N. meningitidis infection, a reduction of risk of occurrence of said infection and/or symptoms associated with said infection.

‘Treatment’ includes both therapeutic treatment and prophylactic or preventative treatment, wherein the object is to prevent or slow down the infection or symptoms of disease. Those in need of treatment include those already with the disorder as well as those prone to have the disorder or those in whom the disorder is to be prevented. The terms ‘therapy’, ‘therapeutic’, ‘treatment’ or ‘treating’ include reducing, alleviating or inhibiting or eliminating the symptoms or progress of a disease, as well as treatment intended to reduce, alleviate, inhibit or eliminate said symptoms or progress.

A further object of the invention is to provide a method of inducing an immune response, in particular a bactericidal immune response, against N. meningitidis, in particular against N. meningitidis of serogroup B, which comprises administering to an individual in need an immunogenic composition according to the invention. Still within the scope of the invention, it is provided a method of treating or preventing a N. meningitidis infection, in particular an infection of N. meningitidis of serogroup B, which comprises administering to a patient in need a composition according to the invention.

In order to achieve the desirable effect, the composition of the invention may be administered as a primary dose, in a primary immunisation schedule, one or several times, e.g., two or three times, at appropriate intervals defined in terms of week or advantageously, month. In a particular embodiment, the interval between the primary doses may be not less than one or two months, depending on the conditions of the subject receiving the doses. If needed, the primary doses may possibly be followed by a booster dose of the composition of the invention, which may be administered e.g., from at least 6 months, preferably at least one year to two-five years, after the last primary dose.

The composition according to the invention may be administered by any conventional routes in use in the vaccine field e.g. by parenteral route such as the subcutaneous or intramuscular route. In a particular embodiment, the composition is suitable for injection and formulated accordingly. It may be in a liquid form or in a solid form that, before administration, may be extemporaneously suspended in a pharmaceutically-acceptable diluent.

Also provided, is a polypeptide of the invention in the manufacture of a medicament for the preventive or therapeutic treatment of a N. meningitidis infection, e.g. an infection of N. meningitidis of serogroup B, such as meningitis.

Also provided, is a polypeptide or composition of the invention for use in a method of inducing an immune response to N. meningitidis, in particular N. meningitidis of serogroup B. Also provided, is a polypeptide or composition of the invention for use in a method of preventing or treating a N. meningitidis infection, e.g. an infection of N. meningitidis of serogroup B, such as meningitis. In some embodiments, said method comprises administering said polypeptide or composition to a subject, in particular a subject in need thereof. According to one embodiment, a method of the invention may comprise the step of observing a preventing and/or a treating effect with regard to a N. meningitidis infection.

Also provided is a method of inducing an immune response to N. meningitidis, in particular N. meningitidis of serogroup B, which comprises administering a polypeptide or composition of the invention to an individual in need thereof. Also provided is a method of preventing or treating of meningitis, in particular N. meningitidis infection, e.g. an infection of N. meningitidis of serogroup B, such as meningitis, which comprises administering a polypeptide or composition of the invention to an individual in need thereof.

The invention will be further illustrated by the following figures, sequences and experimental part:

### BRIEF DESCRIPTION OF THE FIGURES

FIG. 1 shows the amino acid sequence of the full-length NalP and encoding nucleotide sequence (respectively SEQ ID NO: 1 and SEQ ID NO: 2) from N. meningitidis strain MC58. The signal peptide is boxed. Gly30 and Gly853 are underlined, and Ser426 is circled.

FIG. 2 shows the functional domains of NalP. Amino acid numbering is based on the sequence of FIG. 1.

FIG. 3 is a schematic representation of the constructs tested by the inventors.

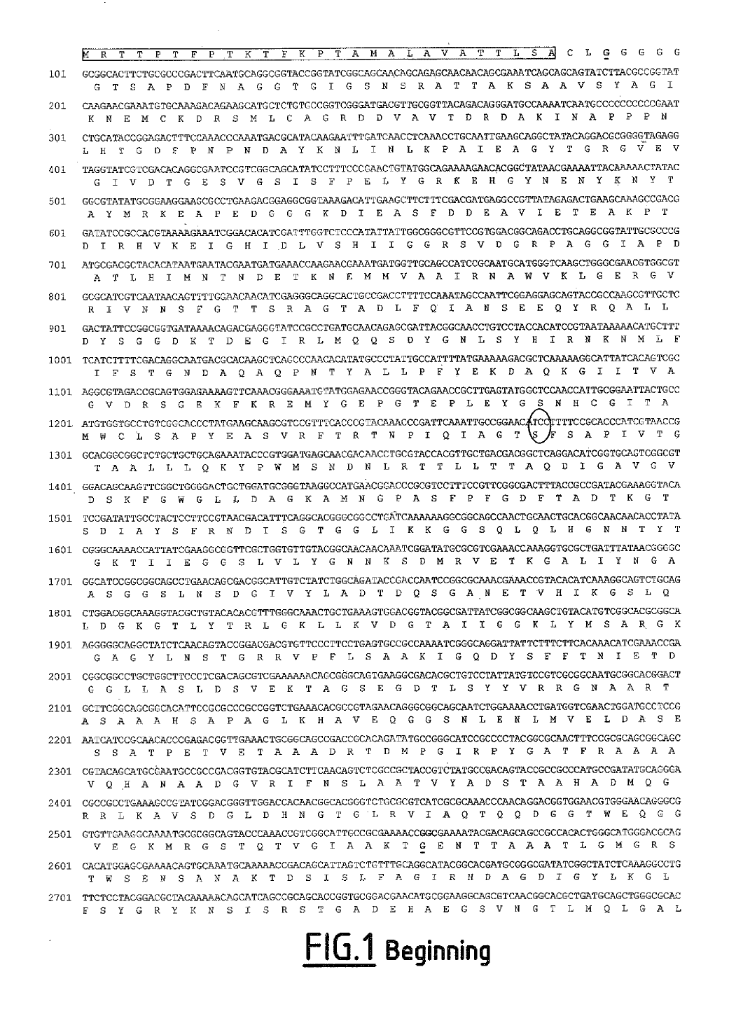

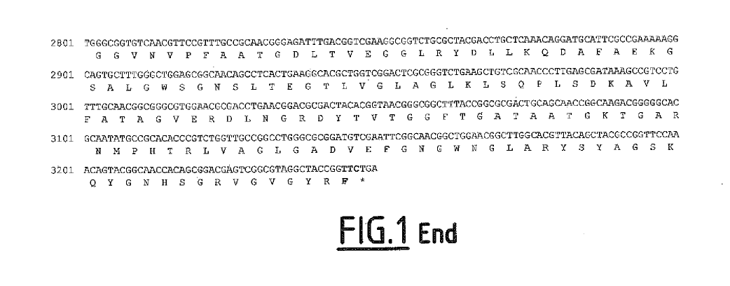

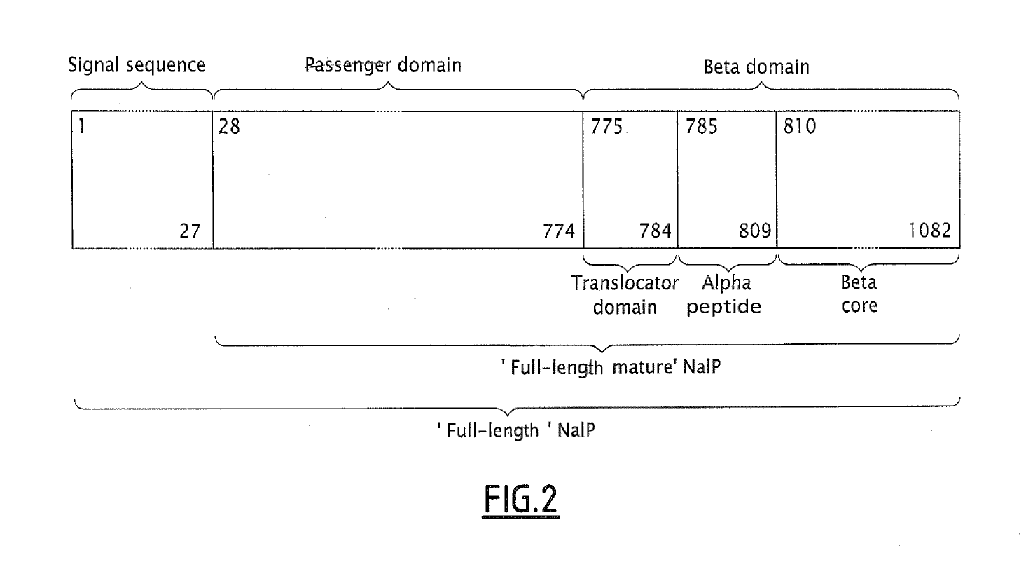

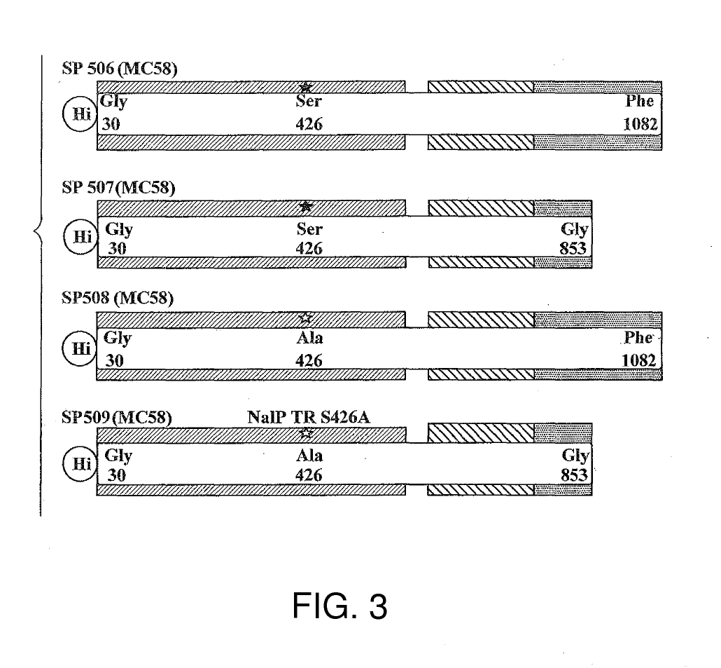

### DESCRIPTION OF THE SEQUENCE LISTING

SEQ ID No:

1 NalP from N meningitidis strain MC58 amino acid sequence

2 NalP from N meningitidis strain MC58 nucleic acid sequence

3 NalP construct SP509 amino acid sequence, without the His-tag

4 NalP construct SP507 amino acid sequence, without the His-tag

5 NalP construct SP508 amino acid sequence, without the His-tag

6 NalP construct SP506 amino acid sequence, without the His-tag

### EXPERIMENTAL

A—Material and Methods

Constructs

The sequence information with respect to the NalP of the N. meningitidis MC58 genome (respectively NMB1969) was retrieved from the Entrez Gene database of the NCBI (National Center for the Biotechnology Information) at http://www.ncbi.nlm.nih.gov under the accession number NC_003112. This sequence information is particularly useful for designing the primers.

The constructs that were produced are as follows:
SP506 (full-length mature NalP) starts with Glycine 30 and ends with Phenylalanine 1082 (amino acid numbering is based on the complete amino acid sequence of NMB1969). A His-tag is added at the N-ter end, without spacer.
SP507 (truncated NalP) starts with Glycine 30 and ends with Glycine 853 (amino acid numbering is based on the complete amino acid sequence of NMB1969). A His-tag is added at the N-ter end, without spacer.
SP508 (full-length mature mutated NalP) starts with Glycine 30 and ends with Phenylalanine 1082 (amino acid numbering is based on the complete amino acid sequence of NMB1969) and comprises the Ser 426 Ala mutation generated by overlap PCR extension. A His-tag is added at the N-ter end, without spacer.
SP509 (truncated mutated NalP) starts with Glycine 30 and ends with Glycine 853 (amino acid numbering is based on the complete amino acid sequence of NMB1969) and comprises the Ser 426 Ala mutation generated by overlap PCR extension. A His-tag is added at the N-ter end, without spacer.

PCR Amplification of SP506 and SP507 ORFs

The genomic DNA of strain MC58 was purified using a purification kit (Roche). SP506 and SP507 ORFs (open reading frame) were amplified by PCR from the purified DNA using appropriate primers and with respectively the Expand Long Template PCR system (Roche) and the Platinum® Taq DNA polymerase High fidelity (Invitrogen) according to the protocol of the supplier. 5′ primers were designed so that a His-tag in introduced as well as restriction sites.

Cloning into Plasmid TOPO Intermediate

The PCR products were cloned into the PCR Blunt TOPO vector (PCR®-TOPO®-BluntII) according to the protocol of the supplier (Invitrogen). The ligation products were transformed into TOP10 competent bacteria supplied with the kit (Invitrogen). The selection of recombinant clones was performed on LB+kanamycin. The plasmids were checked by enzymatic digestion and verification of the restriction profile. Sequencing of inserts validated the plasmids.

Cloning into a pET-28 Plasmid

The SP506 and SP507 ORFs were extracted from the TOPO plasmids by double digestion enzyme NcoI+BamHI. The fragments were isolated by agarose gel migration and cutting the corresponding bands and then purified by electroelution. A plasmid pET-28 (Novagen) stabilized by insertion of a cer element (pET-cer) was prepared using the same protocol. Each ORF was then assembled with pET-cer using T4 DNA ligase (Invitrogen) according to the protocol of the supplier to give pSP506 and pSP507 respectively. The ligation products were transformed into TOP10 competent bacteria. The selection of recombinant clones was performed on LB+kanamycin. The plasmids were checked by enzymatic digestion and verification of the restriction profile. Sequencing of inserts validated the plasmids in which the ORF is placed under the control of the T7 promoter (from pET28).

Transformation into the Expression Strain

Each of pSP506 and pSP507 were used to transform the expression E. coli strain BL21 (DE3) (Novagen) according to the protocol of the supplier. The selection of recombinant clones was performed on LB+kanamycin.

Mutagenesis for Suppressing the Active Catalytic Site 426S

Overlap extension PCR was performed to amplify the ORF to be mutated in two overlapping PCR fragments. The overlapping central primers were designed to introduce the mutation. A third reaction was then used to assemble the first two fragments into one. The reactions were performed with Platinum® Taq DNA Polymerase High Fidelity (Invitrogen) according to the protocol of the supplier. The two overlapping central primers also insert an original restriction site to facilitate the selection of clones carrying the mutation. The mutated ORFs were selected using the original KpnI restriction site created during the mutagenesis and respectively used for substitution into pSP506 and pSP507 to give pSP508 and pSP509.

Each of pSP508 and pSP509 were used to transform the expression strain BL21 (DE3) as described above for pSP506 and pSP507.

Protein Expression and Purification

Cell Culture for Recombinant Expression of MC58 NalP SP506, SP507, SP508 & SP509

BL21 (DE3) E. coli strains transformed by one of the plasmids pSP506-pSP509 were seeded at a ratio 1:500 in Luria Bertani broth (LB) medium supplemented with kanamycin 30 μg/ml and cultured about 3 hrs at 37° C. under stirring (220 rpm) up to a O.D.600 nm of from 0.6 to 0.8. The IPTG is added at 1 mM final. Bacterial cells are harvested by centrifugation and pellets stored at −20° C.

Preparation of MC58 NalP SP506-509 Extracts for Purification on an IMAC Column

The bacterial pellets corresponding to 500 ml of culture are gently washed in PBS pH 8.0 and bacterial suspensions are centrifuged. Pellets are resuspended in PBS pH 8.0, complemented with lysosyme 100 μg/ml, MgCl2 1 mM, Triton X-100 0.1%. Incubation is achieved at 4° C. 15 min under mild stirring.

Benzonase is added at about 1 unit/ml. Suspensions are further incubated at 4° C. 15-30 min; then gently sonicated 1 min in ice and stirred 20 min at 4° C. Suspensions are centrifuged 20 min at 30 000 g, 4° C. Pellets are resuspended in PBS pH 8.0, complemented with Triton X100 0.1% and urea 2 M. Suspensions are incubated for 1 hr at 4° C. under mild stirring and centrifuged 20 min at 30 000 g, 4° C.

The SP506 and 507 pellets are resuspended in Tris-HCl 20 mM, NaCl 300 mM, urea 6 M, pH 8.0. The suspensions are incubated for 1 hr and centrifuged 20 min at 30 000 g 4° C. Supernatants are recovered.

The SP508 and 509 pellets are resuspended in Tris-HCl 20 mM, NaCl 300 mM, Guanidine 6 M and DTT (dithiothreitol) 5 mM, pH 8.0. The suspensions are incubated for 1 hr and centrifuged 20 min at 30 000 g 4° C. Supernatants are recovered.

Purification of MC58 NalP SP506-509

The supernatant SP506 or SP507 is recovered and applied onto an IMAC column (HPLC Biorad Biologic) previously (i) washed with 6 column volumes (CV) of water and (ii) equilibrated with 3 CV of Buffer A (Tris-HCl 20 mM, NaCl 300 mM, urea 6 M, pH 8.0). 6 CV of Buffer A are added. Purification program once the supernatant is applied on the column, is as follows: 6 CV 100% buffer A; 3 CV gradient 100% buffer A→92%+0% buffer B (buffer A+250 mM imidazole)→8%; 6 CV 92% buffer A+8% buffer B; 6 CV gradient 92% buffer A→0%+8% buffer B→100%; 3 CV 100% buffer B.

The purification procedure described above is applied in a similar manner to the supernatant SP508 or SP509, except that urea 6 M is replaced by Guanidine 6 M, DTT 1 mM in buffer A.

Fractions containing SP506-509 were eluted at about 20 mM imidazole. They were pooled and dialyzed overnight against PBS urea 4 M to respectively decrease the urea concentration or remove guanidine. SP506-509 solutions are kept at −80° C.

Before use, each of SP506-509 in PBS urea 4 M is extemporaneously dialyzed against PBS arginine 0.5 M for renaturation.

Immunogenicity, Bactericidal Activity & Flow Cytometry Analysis

Bacterial Strains and Growth Conditions

A set of 26 wild-type serogroup B N. meningitidis strains isolated from geographically distinct locations at different date of isolation and representing diverse MLST clonal complexes were selected for this study. They are listed in Table 3. The majority of the strains were kindly provided by Drs D. A Caugant (NIPH, Norway), D. Martin (EZR, New-Zealand), M. K Taha (IP, Paris), M. A. Diggle (SHLMPRL, Scotland), L. Saarinen (NPHI, Finland).

MenB strains were grown overnight at 37° C. with 10% CO₂ on Brain Heart Infusion (BHI) agar (Difco) plates. Then, the bacteria were harvested from plates and inoculated into BHI broth (Difco) alone or supplemented with or without 30 μM desferal which is a chelator of divalent cations. Cultures were analyzed after 2.5 hrs that correspond to an early exponential growth phase.

Production of Mouse Antisera

To obtain specific immune sera, outbred CD1 mice were immunized 3 times on days 0, 21 and 35, by subcutaneous route, with 10 μg/mouse of the antigen of interest co-injected with adjuvant AF04 [oil-in-water emulsion as described in WO 07/006939, containing the Eisai product ER 804057 (also known as E6020, described in U.S. Pat. No. 7,683,200) as TLR4-agonist. AF04 is described in Examples 1 and 2 of WO 07/080308]. Blood samples were collected on day 42. Blood samples were collected in vacutainer vials containing a coagulation activator and a serum separator gel (BD, Meylan France). Tubes were centrifuged for 20 min at 2600 g in order to separate serum from cells. Sera were transferred into Nunc tubes and heat-inactivated for 30 min at 56° C. They were stored at −20° C. until the assays were performed.

Serum Bactericidal Activity Assay

The bactericidal activity of specific mouse sera was evaluated using as complement source pooled babby rabbit serum as described earlier with slight modifications (Rokbi et al., Clin. Diagnostic Lab. (1997) 4 (5): 522). Briefly, 50 μl of two-fold serial dilutions of IgG solutions or serum were added to 96-well microtiter plates (Nunc) and incubated with 25 μl of a meningococci suspension adjusted to 4×10³ CFU/ml and 25 μl of baby rabbit complement. After 1 hr of incubation at 37° C., 50 μl of the mixture from each well was plated onto MHA plates. The plates were incubated overnight at 37° C. in 10% CO₂. The bactericidal titer (SBA titer) of each serum was expressed as the inverse of the last dilution of serum at which 50% killing was observed compared to the complement control.

The SBA assay is commonly acknowledged as a surrogate of protection for vaccines against N. meningitidis. It is considered that when the SBA titer is superior or equal to 16 in homologous SBA assay, or superior or equal to 8 in heterologous SBA, protection is considered to be met.

Flow Cytometry Analysis

The ability of polyclonal antisera elicited by the recombinant proteins to bind to the surface of live MenB strains was determined using a flow cytometry detection of indirect fluorescence assay. A culture sample was centrifuged and washed once with 1×PBS (Eurobio). The final pellet was resuspended in PBS with 1% of bovine serum albumin (BSA, Eurobio) at a density of 10⁸ CFU/ml. To 20 μl of bacteria, 20 μl of dilutions of pooled serum were added in 96 deep-well plate (Ritter). For each pool of serum, several dilutions were tested on a range going from 1/5 to over 1/2000. The plate was incubated for 1 h at 37° C. with shaking. The bacteria were centrifuged, washed once with PBS 1% BSA and resuspended with 100 μl of goat anti-mouse IgG (H and L chains) conjugated to fluorescein isothiocyanate (FITC) (Southern Biotech) diluted 100-fold. The plate was incubated for 30 minutes at 37° C. with shaking in the dark. The bacteria were washed twice with PBS 1% BSA and fixed with 0.3% formaldehyde in PBS buffer overnight at +4° C. in the dark. The bacteria were centrifuged, the formaldehyde solution was discarded and the bacteria were finally washed once and dissolved in PBS 1% BSA. The fluorescent staining of bacteria was analysed on a Cytomics FC500 flow cytometer (Beckman Coulter). The fluorescent signal obtained for bacteria incubated with the polyclonal antisera or purified IgGs thereof specific for proteins injected with adjuvant was compared to the signal obtained for bacteria incubated with the antisera of mice injected with buffer+adjuvant.

In what follows, the terms “seroconversion”, “seroconversion compared to a negative control” and “fold-increase” are used interchangeably in the text, table and figures.

B—Results

Homologous SBA

The constructs were first assayed for bactericidal activity against the homologous strain MC58. Results are expressed in terms of (i) GMTs (geometric mean titers), (ii) number of responders exhibiting a bactericidal titer superior or equal to 16 and, (iii) fold-increase of GMT compared to the negative control. Results are to be seen in Table 4 below.

TABLE 4
IgG titers and homologous SBA titers (Mouse SBA on MC58 strain for
MC58 NalP constructs)

| | | | Homologous SBA titers | | |
|---|---|---|---|---|---|
| | | | | | Fold-increase |
| | | | | % of responders | compared to |
| | Number | IgG titers | | exhibiting an SBA titer | the negative |
| | of mice | (Log10) | GMT | ≥16 | control |
| SP506 | 9 | 5.8 | 47.0 | 78% | ×20.6 |
| NalP FL | | | | | |
| SP507 | 10 | 5.9 | 17.1 | 40% | ×7.4 |
| NalP TR | | | | | |
| SP508 | 10 | 6.6 | 137.2 | 100% | ×59.7 |
| NalP FL S426A | | | | | |
| SP509 | 10 | 6.4 | 194.0 | 100% | ×84.3 |
| NalP TR S426A | | | | | |
| Control | 10 | | 2.3 | 0% | |
| Adjuvant | | | | | |
| Control PBS | 10 | | 2.6 | 0% | |

FL: full-length
TR: truncated

All forms of NalP are equally immunogenic. With regard to SBA, it is generally considered that a difference is significant when a 4-fold increase in titers is observed by comparison to the negative control (seroconversion). Accordingly, SP507 is a construction of interest as it may be produced and purified more easily than SP506 (full-length NalP). It was not expected that the truncation could lead to SBA results equivalent to those observed with SP506. Considering the SBA results obtained with SP506, SP508 and SP509 altogether, a significant trend towards improvement is to be seen when the mutation S426A is introduced: 100% seroconversion is only observed with constructions bearing the mutation. In addition to this, GMT and seroconversion observed with SP509 which bears the mutation differs from those observed with SP506 by more than a 4-fold increase. In brief, it was not expected that the truncation would not have any detrimental effect and that the mutation would have a beneficial effect on SBA. Further, it was not expected that the truncation and the mutation altogether would lead to the best SBA results.

Heterologous SBA & FACS Analysis

The cross-bactericidal activity of mouse pooled sera raised against the construct leading to the best SBA results (SP509 adjuvanted with the AF04 adjuvant) was further assayed in two independent experiments I and II, against a panel of 25/26 strains (including MC58 and M982). Most of the strains included in this panel are representative of the 5 major epidemiological clusters (ST32, ST11, ST41/44, ST8, ST269). Strains were cultured in BHI+desferal, 2 hr 30 except strain NGH41, cultured for 4 hrs in BHI agar. The presence/absence of the nalP gene in these strains has been previously assessed by PCR. When nalP was found present, the phase variation status ON/OFF was assessed by sequencing. In addition, pools of sera were assessed for their ability to recognize the targeted protein at the surface of viable bacterial cells using flow cytometry (FACS analysis).

SBA results are expressed in terms of fold-increase of GMT compared to a negative control. It is considered that cross-bactericidal activity is met when the fold-increase is superior or equal to 8 (seroconversion). Surface Exposure (SE) is expressed in terms of detection level ranging from [−] to [+++] depending on the highest dilution of the pooled antisera at which surface exposure is detected: [−] at a dilution <1/20e; [+] at a 1/20e dilution: [++] at a 1/200e dilution; and [+++] at a 1/2000e dilution. Results are to be seen in Tables 5 and 6 hereinafter.

Tables 5 and 6 show that out of the 25/26 strains, 15 were able to express the nalP gene (about 60%). This was further confirmed while assessing the surface exposure by flow cytometry analysis (FACS). The expression of the NalP antigen at the bacterial surface as quantified by FACS also tends to correlate with the fold-increase factor measured in SBA.

Importantly, Tables 5 and 6 show that seroconversion (fold-increase >8) occurred against 12 (Experiment II)—13 (Experiment I) out of the 15 strains expressing nalP. It can be therefore concluded that SP509 (MC58 NalP TR mut) is broadly cross-bactericidal against 80-86% of NalP expressing strains.

### REFERENCES

Lomholt et al., Mol. Microb. (1995) 15 (3): 495
Vitovski & Sayers, Infect. Immun. (2007) 75 (6): 2875
Ulsen & Tommassen, FEMS Microbiol. Rev. (2006) 30 (2): 292
WO 90/11367
Tettelin et al., Science (March 2000) 287: 1809
WO 00/26375
Turner et al., Infect. Immun. (2002) 70: 4447
van Ulsen et al., Mol. Microbiol. (2003) 50 (3): 1017
WO 00/66791
Parkhill et al., Nature (March 2000) 404: 502
Bentley et al., PLoS Genet., 3, e23 (2007)
Smith et al., J. Mol. Biol. (1981) 147: 195
Altschul et al., (1990) J. Mol. Biol., 215: 403
Roussel-Jazédé et al., Infect Immun. (2010) 78 (7): 3083
Serruto et al., PNAS Feb. 2010 107 (8): 3770
Sambrook et al., Molecular Cloning: A Laboratory Manual, New York: Cold Spring Harbor Press, 1989
WO 94/00153
U.S. Pat. No. 6,113,918
U.S. Pat. No. 6,303,347
WO 98/50399
US 2003/0153532
WO2007/005583
WO 07/006939
U.S. Pat. No. 7,683,200
WO 07/080308
Rokbi et al., Clin. Diagnostic Lab. (1997) 4 (5): 522

1. An isolated polypeptide comprising or consisting of:
(A) a fragment of a full-length mature N. meningitidis NalP protein, which full-length mature N. meningitidis NalP protein comprises (a) a passenger domain comprising a subtilisin-like serine protease triad (Asp/His/Ser) and (b) a beta-domain comprising a translocator domain, an alpha-peptide and a beta-core composed of twelve beta-sheets; said NalP fragment comprising:
(i) at the N-terminus, the NalP passenger domain;
(ii) at the C-terminus, the beta-domain comprising the translocator domain, the alpha-peptide and at least one and no more than eleven NalP beta-sheets;
or
(B) a mutant of said NalP fragment (A) which lacks or has reduced subtilisin-like serine protease activity and/or does not contain any cleavage site able to be cleaved by a subtilisin-like serine protease;
wherein said polypeptide does not comprise a full-length mature N. meningitides NalP protein.

2. An isolated polypeptide according to claim 1, wherein said polypeptide lacks protease activity, and wherein the NalP passenger domain portion of said polypeptide has an amino acid substitution at the position corresponding to Asp138, His157 or Ser426 of the amino acid sequence of SEQ ID NO:1.

3. An isolated polypeptide according to claim 2, wherein the residue corresponding to Ser426 of SEQ ID NO: 1 is substituted by Ala.

4. An isolated polypeptide according to claim 1, wherein said NalP fragment comprises no more than two NalP beta sheets.

5. An isolated polypeptide according to claim 1, wherein said fragment is a fragment of full-length mature NalP protein of N. meningitidis MC58 or of a NalP variant having at least 95% identity with the NalP protein of N. meningitidis MC58.

6. An isolated polypeptide according to claim 4, wherein said NalP fragment comprises an amino acid sequence which has at least 95% identity with the sequence of SEQ ID NO: 3.

7. An isolated polypeptide according to claim 6, wherein said NalP fragment comprises the amino acid sequence of SEQ ID NO: 3.

8. An isolated polypeptide according to claim 1, wherein
(i) said passenger domain consists of residues at positions 28-774+/−2 amino acids on the C-terminal and/or N-terminal end of said locations of the amino acid sequence of SEQ ID NO:1; and/or
(ii) said beta domain consists of residues at positions 810-1082+/−2 amino acids on the C-terminal and/or N-terminal end of said locations of the amino acid sequence of SEQ ID NO: 1.

9. A composition comprising a polypeptide according to claim 1 or a polypeptide comprising a full-length mature N. meningitidis NalP protein, together with a pharmaceutically acceptable diluent or carrier; wherein said full-length mature N. meningitidis NalP protein lacks subtilisin-like serine protease activity or does not contain any cleavage site able to be cleaved by a subtilisin-like serine protease.

10. A nucleic acid encoding a polypeptide according to claim 1.

11. A vector comprising a nucleic acid according to claim 10.

12. A host cell comprising a nucleic acid according to claim 10.

13. A vaccine composition comprising a polypeptide according to claim 1.

14. (canceled)

15. A vaccine composition comprising a composition according to claim 9.

16. An isolated polypeptide according to claim 5, wherein said NalP fragment comprises an amino acid sequence which has at least 95% identity with the sequence of SEQ ID NO: 3.

17. An isolated polypeptide according to claim 16, wherein said NalP fragment comprises the amino acid sequence of SEQ ID NO: 3.

18. A host cell comprising a vector according to claim 11.

19. A method of treating N. meningitidis B infection in a subject, the method comprising administering an effective amount of a polypeptide according to claim 1.

20. A method of treating N. meningitidis B infection in a subject, the method comprising administering an effective amount of a composition according to claim 9.
